SEQUENCE LISTING (3 sequences)

SEQ ID NO: 1 - DNA, 12 bp, Homo sapiens
  misc_feature: deletion sequence from NALP1 gene
tatgactatgtg

SEQ ID NO: 2 - DNA, 22 bp, Homo sapiens
  misc_feature: primer
tgtgttctcaagtgccttatgc

SEQ ID NO: 3 - DNA, 20 bp, Homo sapiens
  misc_feature: primer
catctttgctcgtgtgtgtg

(19) United States
(12) Patent Application Publication
(10) Pub. No.: US 2009/0298764 A1
(43) Pub. Date: Dec. 3, 2009

(54) GENE AND PATHWAY AND THEIR USE IN METHODS AND COMPOSITIONS FOR PREDICTING ONSET OR PROGRESSION OF AUTOIMMUNE AND/OR AUTOINFLAMMATORY DISEASES

(75) Inventors: Richard A. Spritz, Englewood, CO (US); Ying Jin, Denver, CO (US); Pamela Fain, Golden, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: Regents of The University of Colorado

(21) Appl. No.: 12/282,848

(22) PCT Filed: Mar. 12, 2007
(86) PCT No.: PCT/US07/63833

§ 371 Date: Dec. 5, 2008

Related U.S. Application Data
(60) Provisional application No. 60/782,633, filed on Mar. 15, 2006.

(51) Int. Cl. A61K 38/16; C12N 15/11; C07H 21/04; C12Q 1/68; C12N 5/06

(52) U.S. Cl. 514/12; 536/23.5; 536/24.31; 435/6; 435/375

(57) ABSTRACT

Embodiments of the present invention concern methods, compositions and uses thereof, relating to at least one of vitiligo, or vitiligo-associated autoimmune/autoinflammatory disease (VAAAD). In particular embodiments, genetic variations in the NALP1 gene are of use to detect, diagnose, predict the risk of or treat at least one of vitiligo or VAAAD. In more particular embodiments, the presence of genetic variations such as single-nucleotide polymorphisms (SNPs) in NALP1 genetic region are of use to detect, diagnose or predict the risk of VAAAD. In other embodiments, inhibitors targeted to NALP1, caspase-1 or caspase-5, ASC (PYCARD), interleukin-1β, interleukin-1β receptor, or interleukin 18 may be administered to a subject to treat VAAAD.

### RELATED APPLICATIONS

This application claims the benefit under 35 U.S.C. § 119(e) of U.S. provisional patent application Ser. No. 60/782,633 filed on Mar. 18, 2006 incorporated herein by reference in its entirety.

### FEDERALLY FUNDED RESEARCH

The studies disclosed herein were supported in part by grant numbers R01-AR45584, A146374, DK57538 and U19-AI46374 from the National Institutes of Health. The U.S. government may have certain rights to practice the subject invention.

### FIELD

Embodiments of the present invention relate to methods and compositions of genetic markers for predicting onset or progression of autoimmune and/or autoinflammatory diseases. In other embodiments, methods and compositions of genetic markers for reducing the risk or treatment for autoimmune and/or autoinflammatory diseases are contemplated. Certain embodiments are directed to autoimmune and/or autoinflammatory diseases, including but not limited to, vitiligo, autoimmune thyroid disease, psoriasis, rheumatoid arthritis, adult-onset autoimmune diabetes mellitus (also known as, latent autoimmune diabetes of adults, LADA, type 1.5 diabetes mellitus), pernicious anemia, Addisons's disease and systemic lupus erythromatosis. In certain particular embodiments, the genetic markers can be single nucleotide polymorphisms (SNPs).

### BACKGROUND

Autoimmune and autoinflammatory diseases include a diverse group of approximately 80 disorders that can involve nearly any tissue, organ, or system. Some of these diseases involve specific organs or tissues, whereas with other diseases no particular cell type is specifically targeted. A major source of morbidity and mortality, autoimmune and autoinflammatory diseases collectively affect approximately 15 to 20 million people in the USA. Many autoimmune/autoinflammatory diseases disproportionately affect women and as a group rank among the top ten causes of death of women in all age categories up to 64 years.

The development of autoimmune and autoinflammatory disorders involves complex interactions between genetic risk factors and environmental triggers, some of which appear to be disease-specific, and others of which contribute to susceptibility to many different autoimmune and autoinflammatory diseases. Many patients develop more than one autoimmune/autoinflammatory disease, and various names have been applied to common specific combinations of “multiple autoimmune disease” for example, “Schmidt syndrome”, “endocrine polyglandular syndrome”, “polyglandular autoimmune syndrome type II”, “autoimmune polyendocrine syndrome type II”, and others. Although rare multiple autoimmune disease syndromes result from mutations in single genes for example, AIRE and FOXP3, the great majority of cases do not follow Mendelian patterns of single-gene inheritance and instead represent polygenic, multifactorial disorders. In certain subjects' close relatives also have elevated risks for these same autoimmune/autoinflammatory diseases, suggesting that susceptibility towards these diseases involves shared genetic risk factors. Several genes, such as the HLA class I and II loci, for example, CTLA4, and PTPN22, have been confirmed as risk factors for many individual autoimmune and autoinflammatory diseases, and may also contribute to common forms of multiple autoimmune/autoinflammatory disease. However, these genes have not provided facile targets for therapies aimed at reducing disease risk.

Certain treatments for many autoimmune diseases are relatively non-specific, consisting primarily of symptomatic therapy and large doses of steroids. More specific treatments of autoimmune diseases could concern identification of specific genes and/or genetic variations that are causal for autoimmune disease. Identification of such genes and/or genetic variation could allow development of more targeted therapies directed against the disease inducing pathway or a specific genetic variation providing specific relief for disease treatment and/or prevention.

A need exists for identification of genetic markers that more accurately predict risk of or progression of autoimmune/autoinflammatory disease and/or to identify mutation(s) or polymorphism(s) contributing to such diseases.

### SUMMARY

Embodiments of the present invention provide compositions, methods and uses for predicting risk of, or progression of autoimmune/autoinflammatory disease. In other embodiments, genetic markers are of use to diagnose or to predict the risk of developing autoimmune/autoinflammatory diseases in order to identify therapeutic targets for intervention in autoimmune/autoinflammatory diseases and/or identifying therapeutic agents for treatment of autoimmune/autoinflammatory disease. Certain embodiments concern genetic markers located in or near the NALP1 gene or promoter, of use to predict risk or progression of one or more autoimmune/autoinflammatory disease.

Certain embodiments concern one or more mutations within a particular gene or genetic region, such as single nucleotide polymorphisms of use to predict risk of, or progression of autoimmune/autoinflammatory disease. Some embodiments concern genetic markers such as SNPs or mutations including, but not limited to, rs6502867/A, rs961826/A, rs12150220/A, rs11078575/C, rs1877658/T, rs925597/A, rs925598/A, rs3926687/T, 12 bp deletion, rs2670660/C, rs2733359/G, rs35658367/ATGA, rs2716914/C, rs878329/G, rs7223628/G, rs8182352/G, rs4790796/A, rs4790797/T, rs8182354/A, or combination thereof for use in predicting the onset or risk of autoimmune/autoinflammatory disease in a subject. Some embodiments concern genetic markers such as SNPs or mutations including, but not limited to, rs6502867/A, rs961826/A, rs12150220/A, rs11078575/C, rs1877658/T, rs925597/A, rs925598/A, rs3926687/T, 12 bp deletion, rs2670660/C, rs2733359/G, rs35658367/ATGA, rs2716914/C, rs878329/G, rs7223628/G, rs8182352/G, rs4790796/A, rs4790797/T, rs8182354/A, or combination thereof for use in predicting the onset or risk of vitiligo in a subject.

In other particular embodiments, certain genetic markers contemplated herein can include haplotypes. For example, haplotypes can include but are not limited to, rs-392687-rs2733359-rs878329 (haplotype 1), rs961826-5424661-rs925598-rs8182352-rs4790797-rs8182354 (haplotype 1), rs925598-rs8072203-rs11658667-rs3926687-rs907875-rs2733359-rs2716914-rs7209554-rs878329, or a combination thereof. In addition, other regions contemplated of use for predicting onset of or progression of disease herein include, but are not limited to, identifying other markers adjacent to or within NACHT leucine-rich-repeat protein 1 (NALP1), and genotypes of the component markers which in some cases encompass sections of the 5′ half of the NALP1 structural gene, proximal and distal portions of its extended transcriptional promoter. In accordance with these embodiments, genetic markers or haplotypes can be associated with vitiligo, with vitiligo-associated autoimmune/autoinflammatory disease (VAAAD), or a combination thereof.

In other embodiments, the individual SNPs, or other genetic variations such as haplotypes can be associated with increased or decreased risk of autoimmune disease, and may individually function as genetic markers, or may function as or be in linkage disequilibrium with one or more causal mutations for autoimmune disease, with both therapeutic and diagnostic implications. In one particular embodiment, a coding region SNP at nucleotide position 5402395 with alternative C>G nucleotides results in an amino acid sequence variant Thr783Ser, within a domain referred to as “LRR repeat domain.” Functional SNPs can be of use for detection or diagnostic purposes or may alternatively function as potential therapeutic targets for treatment of subjects with autoimmune/autoinflammatory disease.

In other particular embodiments, a SNP contemplated of use in the present invention may provide more information when combined with other genetic variations such as additional SNPs or haplotypes. In accordance with this embodiment, one exemplary SNP, rs6502867, when used in combination with other genetic variations provides additional information in a subject under study. In certain embodiments, prediction of risk or progression of vitiligo and/or VAAAD in a subject may be assessed by analyzing a sample for a combination of genetic variations in the NALP1 gene region. For example, rs6502867 and at least one other genetic variation may be a more accurate predictor of a condition or progression of a condition than rs6502867 used alone. In other embodiments, compositions and methods herein may include the use of 2 or more, 3 or more, 4 or more, 5 or more, 6 or more, 7 or more, 8 or more, 9 or more, 10 or more of genetic variations disclosed herein. In some embodiments, certain methods and compositions can include detection of genetic variations of one or more of rs961826, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2733359, rs35658367, rs2716914, and rs8182352; and one or more of rs2670660, rs878329, rs7223628, rs8182352, rs4790796, and rs4790797. In an even more particular embodiment, genetic variations of the NALP1 region of use herein can include rs6502867 and at least one of rs961826, rs12150220, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO:1) or their reverse complement), rs2670660, rs2733359, rs35658367, rs2716914, rs878329, rs7223628, rs8182352, rs4790796, rs4790797, or rs8182354.

Certain embodiments may concern probes, primers and/or kits of use to detect the genetic markers associated with risk of or progression of autoimmune/autoinflammatory disease. Other embodiments concern methods of use for probes, primers and/or kits to detect the presence of and/or determine the risk of developing autoimmune/autoinflammatory disease. In particular embodiments, the probes, primers and/or kits may be designed to detect a genetic polymorphisms disclosed above. In alternative embodiments, antibodies or other binding molecules (e.g., aptamers) that selectively or specifically recognize the protein product of the NALP1 gene may also be of use to predict the risk of or progression of autoimmune/autoinflammatory disease.

Once a putative causative gene/genetic variation has been determined, it may serve as a target for therapeutic intervention in the disease process. For example, if overexpression of a given gene is associated with increased risk of disease, then expression may be inhibited, for example by anti-sense or RNA interference technologies well known in the art. (See, e.g., PCT Patent Application Publication No. WO 01/75164; PCT Patent Application Serial No. PCT/EP01/13968; U.S. patent application Ser. Nos. 09/821,832; 10/255,568, each incorporated herein by reference.)

Alternatively, the protein product of a putative causal gene may be targeted for development of inhibitors/agonists, including but not limited to antibodies, antibody fragments, antibody analogs, small molecule therapeutics, etc. Methods of antibody/fragment production are well known in the art, as discussed below. Various techniques for developing small molecule therapeutics, such as high-throughput screening, are also known in the art. (See, e.g., Doman et al., 2002, J. Med. Chem. 45:2213-21; Bajorath, 2002, Nature Rev. Drug Discovery 1:882-894; Handbook of Drug Screening, Seethala and Fernandes (eds), 2001, Marcel Dekker, New York, N.Y.).

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1A represents an exemplary linkage map on Chromosome 17 for vitiligo-associated multiple autoimmune/autoinflammatory disease, based on microsatellite markers.

FIG. 1B represents an exemplary SNP genotyping linkage map for 177 SNPs genotyped across the 17p linkage region (0.04 to 6.24 Mb) in 51 multiplex families with vitiligo-associated multiple autoimmune/autoinflammatory disease; 23 SNPs were significantly associated with disease in these 51 families, including three blocks of multiple contiguous SNPs with significant (p≦0.05) or almost-significant individual p-values, for both vitiligo alone and for the entire group of vitiligo-associated autoimmune diseases, were observed. These 23 SNPs were then subsequently genotyped in an independent group of 63 multiplex families with vitiligo-associated autoimmune/autoinflammatory disease; a block of three contiguous SNPs with significant (p≦0.05) p-values, for both vitiligo alone and for the entire group of vitiligo-associated multiple autoimmune/autoinflammatory diseases was observed.

FIG. 1C represents an exemplary fine mapping study of the NALP1 region in a 114-family cohort, genotyping a total of 85 SNPs distributed across the NALP1 gene and 246 kbp of its adjacent 5′ promoter region for allelic and haplotype association with vitiligo alone and with vitiligo-associated multiple autoimmune/autoinflammatory disease.

FIG. 1D represents an exemplary detailed map of linkage disequilibrium patterns for the 19 SNPs distributed across the NALP1 gene and 246 kbp of its adjacent 5′ promoter region that were most consistently associated with disease.

### DESCRIPTION OF ILLUSTRATIVE EMBODIMENTS

### Definitions

Terms that are not otherwise defined herein are used in accordance with their plain and ordinary meaning.

As used herein, “a” or “an” may mean one or more than one of an item.

As used herein, a “genetic marker” may refer to any genetic variation that appears to be linked to, or to be predictive of risk for, a given phenotype (trait) or group of traits. Genetic markers may include, but are not limited to, single nucleotide polymorphisms (SNPs), insertions, deletions, substitutions, mutations, RFLPs (restriction fragment length polymorphisms), VNTRs (variable number of tandem repeats), STRs (short tandem repeats), microsatellite sequences, hapoltypes or any other type of known genetic variation or combination thereof. A genetic marker is of use if it is associated with either an increased or decreased probability of exhibiting a phenotype. Although a statistically significant degree of association between marker and phenotype is preferred, in some embodiments markers may be of use even if the association with phenotype is not statistically significant.

### Vitiligo-Associated Multiple Autoimmune/Autoinflammatory Disease

Multiple autoimmune disease is a term used to describe common association of various autoimmune diseases, both in individual patients and their relatives. A strong epidemiologic association among a specific group of autoimmune/autoinflammatory diseases that includes generalized vitiligo, autoimmune thyroid disease (Graves' disease and autoimmune hypothyroidism), adult-onset autoimmune diabetes mellitus, rheumatoid arthritis, psoriasis, pernicious anemia, systemic lupus erythematosus, and Addison disease has been shown. Vitiligo has been used as a signal disorder for this group due to its ease of clinical diagnosis and high heritability. By genetic linkage analysis of multiple autoimmune disease families that segregate both generalized vitiligo and these other associated autoimmune/autoinflammatory diseases, this group of disorders has been shown to be genetically linked to the short arm of chromosome 17 (17p).

Researchers have mapped a locus (SLEV1) that confers susceptibility to autoimmune/autoinflammatory diseases (Table 1) that include generalized vitiligo, autoimmune thyroid disease (Graves' disease and autoimmune hypothyroidism), adult-onset autoimmune diabetes mellitus, rheumatoid arthritis, psoriasis, pernicious anemia, systemic lupus erythematosus, and Addison disease to chromosome segment 17p13. By fine-scale genetic association and DNA sequence analyses of multiplex families with vitiligo-associated multiple autoimmune/autoinflammatory disease, the gene corresponding to SLEV1 as NALP1, encoding a key regulator of the innate immune system was identified.

### NALP 1

NACHT leucine-rich-repeat protein 1 (NALP1) also known as CARD7, DEFCAP, and NAC, is thought to mediate activation of the innate immune system in response to pathogenassociated molecular patterns such as bacterial peptides. NALP1 is widely expressed at low levels but is expressed at a high level in immune cells, particularly T cells and Langerhans' cells, patterns that are consistent with the particular involvement of NALP1 in for example, skin autoimmunity. NALP1 recruits the adapter protein ASC, caspase 1, and caspase 5 to a complex termed the NALP1 inflammasome, which activates the proinflammatory cytokine interleukin-1β. Serum interleukin-1β levels are elevated in patients with generalized vitiligo, suggesting the involvement of this pathway in the pathogenesis of disease. NALP1 also appears to play a role in cellular apoptosis, its overexpression stimulating caspase-mediated apoptosis in a variety of cell types. Mutations in at least two other NALP-related genes involved in the innate immune system are associated with autoinflammatory diseases.

NALP1 interacts with ASC, caspase-1, and caspase-5 to form a complex termed the ‘inflammasome’, promoting processing of interleukin-1β (IL-1β) and perhaps IL-18, thereby initiating the autoimmune/autoinflammatory response. Over-expression of NALP1 induces cellular apoptosis, possibly via an APAF-1 apoptosome-mediated mechanism. In certain embodiments contemplated herein, these pathways offer drug targets for reducing the occurrence or progression of the autoimmune/autoinflammatory diseases. Other embodiments concern analysis of the NALP1 and/or NALP1 promoter genetic region for specific pharmacogenetic testing of autoimmune disease susceptibility and/or therapeutic intervention. In accordance with these embodiments, therapeutic intervention can include reducing the risk for autoimmune/autoinflammatory diseases and/or treatment of subjects identified as having autoimmune/autoinflammatory diseases or at risk thereof.

NALP1 is highly expressed in cells of the immune system and is a member of the NBS (nucleotide-binding site) family of proteins that have important roles in caspase activation and apoptosis. NALP1 had been reported to form part of an inflammasome complex with caspase-1 and caspase-5 that is involved in processing of the precursor to the mature form of IL-1β. Because of the genetic linkage data reported herein for the association of NALP1 genetic variations with autoimmune/autoinflammatory disease, and the known involvement of NALP1 in caspase function, NALP1 may function as a “master switch” to regulate the activity of the caspase pathway in various disease/cellular processes, such as autoimmune/autoinflammatory diseases and apoptosis. The NALP1-mediated autoimmune/autoinflammatory response offers an attractive target for drugs that block activity of cellular caspases and the inflammatory and apoptotic pathways controlled by NALP1.

Previous reports disclosed genetic linkage between SLEV1, a linkage signal mapped to 17p13, and occurrence of systemic lupus erythematosus in families that also had at least one case of vitiligo, but no specific genes associated with and/or causal for systemic lupus erythematosus, vitiligo, or vitiligo-associated multiple autoimmune/autoinflammatory disease were demonstrated. Prior to the present disclosure, the underlying gene(s) for this complex of diseases was unknown.

Many patients eventually have more than one autoimmune or autoinflammatory disease. Various names have been applied to different combinations of multiple autoimmune disease, such as Schmidt's syndrome and autoimmune polyglandular syndromes. A few multiple autoimmune disease syndromes result from mutations in single genes; however, most cases of multiple autoimmune disease do not follow mendelian patterns of inheritance, but rather have complex inheritance patterns. Susceptibility genes in these cases may fall into two categories: some may specifically predispose patients to one or more of the component diseases, whereas others may affect the susceptibility of patients to autoimmune and autoinflammatory disease in general. The latter type of gene may represent a target in the treatment or even prevention of several different diseases.

It has been observed among patients with generalized vitiligo that there is an increased frequency of several other autoimmune and autoinflammatory diseases, particularly autoimmune thyroid disease (Graves' disease and autoimmune hypothyroidism), latent autoimmune diabetes in adults, rheumatoid arthritis, psoriasis, pernicious anemia, systemic lupus erythematosus, and Addison's disease. There is also an increased frequency of these same disorders among first-degree relatives of patients with vitiligo, suggesting that some families have a genetic predisposition to this group of autoimmune and autoinflammatory diseases.

By testing for genetic linkage between disease and polymorphic DNA markers spanning the whole genome in families with vitiligo and other autoimmune and autoinflammatory diseases (Teble 1), several chromosomal regions (or loci) have been identified that appear to contribute to this epidemiologic association, including one on chromosome 17p13. This genomic region also appears to contribute to systemic lupus erythematosus in members of families who inherit lupus together with either vitiligo or various other autoimmune and autoinflammatory diseases. This finding suggests that chromosome 17p13 is involved in the susceptibility to multiple autoimmune disease. Fine-scale genetic association and DNA sequence analyses was performed in the 17p13 region in 114 families with vitiligo and associated autoimmune and autoinflammatory diseases.

In certain embodiments, genetic association between the autoimmune/autoinflammatory diseases and single-nucleotide variants located in and around the NALP1 region are contemplated. As shown in the Examples, by fine-scale SNP allelic association and DNA sequence analyses of multiplex families with vitiligo-associated multiple autoimmune/autoimmunity disease, NALP1 was identified as the 17p multiple autoimmunity susceptibility gene. Other embodiments herein concern specific NALP1 SNPs or SNP haplotypes that confer elevated risk of disease and/or disease progression. In addition, specific sequence variation(s) in the NALP1 structural gene and the extended NALP1 transcriptional promoter are also contemplated to mediate susceptibility to autoimmune/autoinflammatory disease. In accordance with these embodiments, genetic testing for susceptibility to this group of diseases can be performed by standard methods known in the art. In addition, directed pharmacologic therapy and directed pharmacologic disease prevention prophylaxis in a subject demonstrated to be at risk for the autoimmune/autoinflammatory disease or disease progression can be administered.

In other embodiments, the NALP1 genetic region may thus provide the opportunity for both specific pharmacogenetic testing for autoimmune/autoinflammatory disease susceptibility and specific pharmacologic intervention to prevent or treat disease in patients at elevated genetic risk. By analogy to rare single-gene genetic diseases involving a related gene, NALP3, mutations in NALP1 may be treatable or preventable by use of inhibitors of distal members of the NALP-dependent autoinflammatory pathway. Inhibitors of downstream members of a NALP1-dependent autoinflammatory pathway, such as CASP1, CASP5, or other caspases (e.g., ICEBERG, pseudo-ICE, N-acetyl-Asp-Glu-Val-Asp-aldehyde (DEVD-CHA), acetyl-Tyr-Val-Ala-Asp-chloromethylketone (AC-YVAD-CMK), t-butoxycarbonyl-L-aspartic acid benzyl ester-chloromethylketone (Boc-Asp-(OBzl)-CMK)), may provide therapeutic treatment for vitiligo-associated multiple autoimmune/autoinflammatory disease (VAAAD). Other pharmacologic approaches that modulate the NALP1-mediated inflammatory and apoptotic pathways in persons at specific genetic risk due to NALP1 may also be possible.

These findings have numerous implications for application, including but not limited to: 1) Genetic testing for genomic variation in or adjacent to the NALP1 gene that determines susceptibility to multiple autoimmune diseases (individually or in combination), including but not limited to, vitiligo, autoimmune thyroid disease, psoriasis, rheumatoid arthritis, diabetes mellitus, pernicious anemia, Addison's disease, and systemic lupus erythematosus. These analyses can include assessing risk or lack of risk for disease or disease progression. 2) Functional testing for dysfunction or activation of the CASP-mediated autoinflammatory pathway or its components in patients with or at risk for these autoimmune/autoinflammatory diseases. 3) Use of inhibitors of the CASP autoinflammatory pathway for pharmacologic treatment of autoimmune diseases associated with NALP1-mediated disease susceptibility. 4) Use of inhibitors of the CASP inflammatory to prevent occurrence or recurrence of autoimmune diseases associated with NALP1-medicated disease susceptibility. 5) Use of other pharmacologic agents to up-regulate or down-regulate the NALP1-mediated inflammatory and apoptotic pathways in persons at specific genetic risk due to NALP1, as appropriate.

In certain embodiments, autoimmune/autoinflammatory diseases may include, but are not limited to, Addison's disease, allergies/asthma/eczema, ankylosing spondylitis (Strümpell-Marie disease/Bekhterev-Strümpel syndrome/spondyloarthritis), antiphospholipid syndrome (anti-cardiolipin syndrome), APECED syndrome (autoimmune polyendocrine syndrome type 1; APS1), autoimmune hemolytic anemia, autoimmune hepatitis (non-infectious chronic active hepatitis), Behçet's disease, bullous pemphigoid, cardiomyopathy, celiac disease (celiac sprue/gluten enteropathy), chronic inflammatory demyelinating polyneuropathy, Churg-Strass syndrome (allergic granulomatosis), cicatrical pemphigoid (mucous membrane pemphigoid/benign pemphigoid), CREST syndrome, cold agglutinin disease, diabetes (including insulin-dependent diabetes mellitus/type 1/juvenile diabetes, non-insulin-dependent diabetes mellitus/type 2/adult-onset), essential mixed cryoglobulinemia, fibromyalgia-fibromyositis syndrome, Guillain-Barré syndrome, IgA nephropathy, idiopathic thrombocytic purpura (ITP), inflammatory bowel disease (Crohn's disease/ulcerative colitis/irritable bowel syndrome), hypo- or hyper-parathyroidism, kidney disease (glomerulonephritis/nephrosis/nephritic syndrome), juvenile arthritis, lichen planus, multiple sclerosis, myasthenia gravis, pernicious anemia, pemphigus vulgaris, polyarteritis nodosa, polychondritis, polymyalgia rheumatica, polymyositis and dermatomyositis (PM_DM syndrome), primary biliary cirrhosis, psoriasis, Raynaud's phenomenon, Reiter's syndrome, rheumatic fever, rheumatoid arthritis, sarcoidosis, Schmidt syndrome (autoimmune polyendocrine syndrome type 2; APS2), Sjogren's syndrome, Stiff-man syndrome (Moersch-Woltmann syndrome), Takayasu arteritis, temporal arteritis (giant cell arteritis), thyroid disease (Graves disease/myxedema/hyperthyroidism, Hashimoto's thyroiditis/goiter/hypothyroidism), uveitis, and vitiligo.

In more particular embodiments, autoimmune/autoinflammatory diseases of interest may include, but are not limited to, vitiligo, autoimmune viriod, rheumatoid arthritis, thyroid disease (Graves disease/myxedema/hyperthyroidism, Hashimoto's thyroiditis/goiter/hypothyroidism), latent autoimmune diabetes of adults, LADA, Type 1.5 diabetes, pernicious anemia, psoriasis, systemic lupus erythematosus (lupus; SLE), Grave's disease, chronic autoimmune diseases or a combination thereof.

Other embodiments concern compositions and methods of use to detect and/or identify mutations/polymorphisms in or near the NALP1 gene that predict the onset of or progression of autoimmune/autoinflammatory disease. Techniques known in the art are contemplated for this purpose. For example, where a defined SNP and/or haplotype is found to be associated with increased or decreased risk of disease, samples can be obtained from a subject known to have the disease and/or relatives of the subject. Haplotypes can be sequenced in a subject and compared to haplotype sequences in a control group of unaffected control subjects. Genetic variations identified by sequence analysis or other methods may be examined for co-inheritance with the disease phenotype. Those genetic variations that exhibit co-inheritance with a disease susceptibility or disease protective phenotype are putatively causal. Causality may be confirmed by enzymatic activity, binding or other biochemical analyses. Where such genetic variations occur in a promoter, enhancer, transcription factor binding site or other gene regulatory sequence, their effect on gene expression may be assayed.

In certain embodiments, compositions contemplated herein concern oligonucleotides including at least a portion of a nucleic acid sequence of the NALP1 gene, NALP1 promoter region or combination thereof. In accordance with these embodiments, compositions herein can include at least a portion of a nucleic acid sequence of the NALP1 gene, NALP1 promoter region or combination thereof, including one or more genetic variations associated with vitiligo or vitiligo-associated autoimmune/autoinflammatory disease (VAAAD) selected from the group consisting of rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO:1)), rs2670660/C, rs2733359/G, rs35658367/ATGA, rs2716914/C, rs878329/G, rs7223628/G, rs8182352/G, rs4790796/A, rs4790797/T, rs8182354/A, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’ haplotypes; any SNP listed in Table 9; any two-SNP haplotype as listed in Table 11 or the reverse complement of any of these variations, and a combination thereof.

In some embodiments, more particular SNPs are contemplated of use for predicting the increased or decreased risk for onset or progression of vitiligo and/or VAAAD in a subject. In certain particular embodiment, the genetic variations can include one or more of rs3926687, rs2733359 and rs878329. In one example, a genetic variation can include a haplotype of SNPs for example, Haplotype 1 that includes at least rs3926687, rs2733359 and rs878329. Other combination genetic variations are contemplated of use in methods and compositions disclosed herein for example, genetic variations can include rs470797 and one or more of rs878329, rs7223628, rs8182352, or genetic variations include rs4790796; rs2670660/C, rs878329/G, re7223628/G, rs8182352/G, rs4790797/T or combination thereof; or genetic variations can include one or more of rs961826, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2733359, rs35658367, rs2716914, and rs8182352; and one or more of rs2670660, rs878329, rs7223628, rs8182352, rs4790796, and rs4790797; or genetic variations can include rs6502867 and at least one of rs961826, rs12150220, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2670660, rs2733359, rs35658367, rs2716914, rs878329, rs7223628, rs8182352, rs4790796, rs4790797, or rs8182354; or the genetic variations can include rs6502867; and at least one of rs12150220, rs2670660, rs878329, rs7223628, rs8182352, rs4790796, or rs4790797; or the genetic variations can include rs6502867, rs12150220 or combination thereof. In a more particular embodiment, genetic variations of use herein can include rs6502867, rs12150220 or combination thereof. In one example, a genetic variation including rs12150220 may include a particular substitution of histidine for leucine at position 155 (Leu155→His).

In some embodiments, a nucleic acid sequence of use for generating a probe or otherwise may contain 2 or more, 3 or more, 4 or more, 5 or more, 6 or more, 7 or more, 8 or more, 9 or more, 10 or more, 11 or more, 12 or more, 13 or more, 14 or more, 15 or more of the genetic variations disclosed herein.

In other embodiments, a probe is contemplated for use in methods and compositions disclosed herein. One particular embodiment, concerns a probe, including but not limited to, an oligonucleotide capable of binding at least a portion of a nucleic acid sequence of the NALP1 gene, NALP1 promoter region or combination thereof concluding, one or more genetic variations associated with vitiligo or vitiligo-associated autoimmune/autoinflammatory disease (VAAAD) selected from the group consisting of rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO:1)), rs2670660/C, rs2733359/G, rs35658367/ATGA, rs2716914/C, rs878329/G, rs7223628/G, rs8182352/G, rs4790796/A, rs4790797/T, rs8182354/A, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’ haplotypes; any SNP listed in Table 9; any two-SNP haplotype as listed in Table 11 or the reverse complement of any of these variations, and a combination thereof. In a more particular embodiment, genetic variations for detection by a probe can include one or more of rs3926687, rs2733359, and rs878329, rs12150220 with a substitution of histidine for leucine at position 155 (Leu155→His) or a complementary sequence thereof, or a probe capable of binding to one or more of rs6502867; and at least one of rs12150220, rs2670660, rs878329, rs7223628, rs8182352, rs4790796, or rs4790797. In certain more particular embodiments, a probe contemplated of use may include a region capable of recognizing one or more of the genetic variations disclosed herein. In accordance with these embodiments, one or more samples may be obtained from a subject suspected of having or at risk for vitiligo and/or VAAAD and the samples analyzed for the presence of a particular genetic variation. Increased or decreased risk for or progression of vitiligo or VAAAD can be assessed. Techniques known by the skilled artisan can be used to obtain samples, analyze the samples and assess the likelihood for these diseases. Information obtained from these analyses may be used in order to assess the need for therapeutic intervention for relieving a symptom of a disease, preventing onset and/or progression of a disease.

Certain embodiments concern method for assessing the risk of at least one of vitiligo or vitiligo-associated autoimmune/autoinflammatory disease (VAAAD) including detecting the presence of one or more genetic variations in the NALP1 gene, NALP1 promoter region or combination thereof. In certain embodiments, methods include detection of one or more genetic variations, including but not limited to, rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1)), rs2670660/C, rs2733359/G, rs35658367/ATGA, rs2716914/C, rs878329/G, rs7223628/G, rs8182352/G, rs4790796/A, rs4790797/T, rs8182354/A, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’ haplotypes; any SNP listed in Table 9; any two-SNP haplotype as listed in Table 11 or the reverse complement of any of these variations, the complement thereof and a combination thereof. In certain particular embodiments, methods concern analyzing a sample from a subject for genetic variations including rs6502867; and at least one of rs12150220, rs2670660, rs878329, rs7223628, rs8182352, rs4790796, or rs4790797. In other embodiments, the risk of vitiligo or VAAAD can be increased or decreased depending on the presence or absence of certain genetic variations for example, rs961826, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2733359, rs35658367, rs2716914, and rs8182352; and one or more of rs2670660, rs878329, rs7223628, rs8182352, rs4790796, and rs4790797.

In other embodiments, method for diagnosing at least one of vitiligo or any of the component disorders of VAAAD are contemplated for example detecting the presence of one or more genetic variations in the NALP1 gene, NALP1 promoter region or combination thereof, the variations associated with risk of VAAAD. In one particular example, detection of one or more genetic variations can include rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2670660/C, rs2733359/G, rs35658367/ATGA, rs2716914/C, rs878329/G, rs7223628/G, rs8182352/G, rs4790796/A, rs4790797/T, rs8182354/A, or the reverse complement of any of these variations, and a combination thereof. In one particular embodiment, genetic variations detected in a sample can include rs6502867; and at least one of rs12150220, rs2670660, rs878329, rs7223628, rs8182352, rs4790796, or rs4790797.

Other more particular embodiments concern a method for assessing the risk of adult-onset autoimmune diabetes mellitus (also known as latent autoimmune diabetes in adults, LADA, and type 1.5 diabetes mellitus) in a subject includes detecting the presence of one or more genetic variations in the NALP1 gene, NALP1 promoter region or combination thereof, the variations associated with risk of at least one of adult-onset autoimmune diabetes mellitus or any of the component disorders of VAAAD. In one exemplary embodiment, one or more samples from a subject may be assessed for the presence or absence of one or more genetic variations, including but not limited to, rs6502867, rs8074853, rs16954840, rs12150220, rs2670660, rs8182352, rs11078587, rs1008588 and a combination thereof. In other embodiments, analysis of a sample from a subject may include genetic variations including, but not limited to, rs6502867; and at least one of rs12150220, rs2670660, rs878329, rs7223628, rs8182352, rs4790796, or rs4790797.

Other methods contemplated herein include, treating a subject for at least one of vitiligo or any of the component disorders of VAAAD including administering to the subject in need of such a treatment, a therapeutically effective amount of a composition targeting one or more genetic variations in NALP1 gene, NALP1 promoter region or combination thereof. Therapeutic treatments contemplated herein may include targeting certain genetic variations in NALP1 gene, NALP1 promoter region or combination thereof including, but not limited to, rs6502867, rs961826, rs12150220, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2670660, rs2733359, rs35658367, rs2716914, rs878329, rs7223628, rs8182352, rs4790796, rs4790797, rs8182354, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’ haplotypes; any SNP listed in Table 9; any two-SNP haplotype as listed in Table 11 or the reverse complement of any of these variations, and a combination thereof. In certain specific embodiments, a composition contemplated herein may ameliorates a symptom of at least one of vitiligo or any of the component disorders of VAAAD.

Examples of symptoms caused by or as a result of vitiligo or component disorders of VAAAD contemplated herein include (but are not limited to) acquired depigmentation of skin and/or overlying hair and/or mucous membranes, fatigue, cold intolerance, bradycardia, dry skin, weight gain, proptosis, weight loss, palpitations, tachycardia, alopecia, nausea, vomiting, depression, hyperpigmentation of skin or mucous membranes, rash, nephritis, arthritis, joint contractures, discoid skin lesions, polyuria, polydipsia, diabetic ketoacidosis, hyperglycemia, megaloblastic anemia, scaling skin lesions, and skin erythema.

In other examples, a therapeutic treatment contemplated herein may include administering a therapeutically acceptable amount of a composition that reduces the risk of developing or reduces the risk of progression of at least one of vitiligo or any of the component disorders of VAAAD. In other examples, therapeutic compositions contemplated of use herein may further comprise an interleukin-1β inhibitor, an interleukin-1β receptor antagonist (IL1-RA, Kinaret, Anikinra) or interleukin-1β receptor inhibitor, a caspase inhibitor, an interleukin-18 antagonist, an interleukin-18 inhibitor, an ASC (PYD and CARD domain containing protein; PYCARD) antagonist, an ASC inhibitor, or combination thereof.

Certain embodiments herein include a method of inhibiting apoptosis including administering a therapeutic agent targeted to NALP1 or a caspase, wherein said administering inhibits apoptosis. In accordance with these embodiments, an agent can be administered to one or more cells, or to a subject. A subject of the present invention can include a human or a non-human subject. In certain preferable embodiments, the subject is a human.

### Nucleic Acids

Various embodiments may concern isolated nucleic acids encoding proteins that confer or are associated with a trait. In other embodiments, the nucleic acid itself may confer or be associated with a trait. In certain embodiments, nucleic acid genetic variations, such as SNPs, mutations, insertions or deletions, may be of use for detection, diagnosis or prognosis of various disease states, such an autoimmune/autoinflammatory disease. Isolated nucleic acids of use for such applications may include primers and/or probes selective or preferably specific for such diagnostic genetic variations. In other embodiments, specific inhibitors that are targeted to the nucleic acid or protein products of the NALP1 gene, or to disease-associated variations in the NALP1 gene, may be of use for therapeutic treatment of such diseases.

As used herein, isolated nucleic acid may be derived from genomic DNA, complementary DNA (cDNA) or synthetic DNA. A “nucleic acid” includes single-stranded and double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39,40,41,42,43,44,45,46,47,48,49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length, up to a full length protein encoding or regulatory genetic element.

Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a trait conferring or trait associated genetic element. For example, a multi-cloning site comprising one or more endonuclease restriction sites may be added. Regulatory sequences may be added to promote expression of the nucleic acid. A nucleic acid may be attached to a vector, adapter, or linker for cloning and/or expression of a nucleic acid. Additional sequences may be added to such cloning and/or expression sequences to optimize their function in cloning and/or expression, to aid in isolation of the nucleic acid, or to improve the introduction of the nucleic acid into a cell. Use of cloning vectors, expression vectors, adapters, and linkers is well known in the art.

Synthetic Methods for Constructing Nucleic Acids

Isolated nucleic acids may be prepared by direct chemical synthesis by methods such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetra. Lett. 22:859-1862 (1981); the solid phase phosphoramidite triester method of Beaucage and Caruthers, Tetra. Letts. 22(20):1859-1862 (1981), using an automated synthesizer as in Needham-VanDevanter et al., Nucleic Acids Res., 12:6159-6168 (1984); or by the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. While chemical synthesis of DNA is best employed for sequences of about 100 bases or less, longer sequences may be obtained by the ligation of shorter sequences. Synthetic oligonucleotides may be of use, for example, for probes and/or primers designed to be used to detect the presence or absence of target nucleic acid sequences, such as the genetic variations in NALP1 associated with autoimmune/autoinflammatory disease. Recombinant Methods for Constructing Nucleic Acids

Isolated nucleic acids may be obtained from genomic or cDNA libraries or other sources using any number of cloning methodologies known in the art. In some embodiments, oligonucleotide probes which selectively hybridize, under stringent conditions, to the nucleic acids are used to identify a sequence in a genomic DNA library. Methods for construction of genomic libraries are known and any such known methods may be used. [See, e.g., Current Protocols in Molecular Biology, Ausubel, et al., Eds., Greene Publishing and Wiley-Interscience, New York (1995); Sambrook, et al., Molecular Cloning: A Laboratory Manual, 2nd Ed., Cold Spring Harbor Laboratory Vols. 1-3 (1989); Methods in Enzymology, Vol. 152, Guide to Molecular Cloning Techniques, Berger and Kimmel, Eds., San Diego: Academic Press, Inc. (1987).]

Nucleic Acid Screening and Isolation

Sources of genomic or cDNA sequences, such as samples from a patient, may be screened for the presence and/or expression levels of an identified genetic element of interest using a probe based upon one or more sequences. Various degrees of stringency of hybridization may be employed in the assay. As the conditions for hybridization become more stringent, there must be a greater degree of complementarity between the probe and the target for duplex formation to occur. The degree of stringency may be controlled by temperature, ionic strength, pH and/or the presence of a partially denaturing solvent such as formamide. For example, the stringency of hybridization is conveniently varied by changing the polarity of the reactant solution through manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100 percent; however, minor sequence variations in the probes and primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture. Nucleic acids may be completely complementary to a target sequence or may exhibit one or more mismatches.

Nucleic Acid Amplification

Nucleic acids of interest may also be amplified using a variety of known amplification techniques. For instance, polymerase chain reaction (PCR) technology may be used to amplify target sequences directly from genomic DNA or cDNA. PCR and other in vitro amplification methods may also be useful, for example, to clone nucleic acid sequences that code for proteins to be expressed, to make nucleic acids to use as probes for detecting the presence of a target nucleic acid in samples, for nucleic acid sequencing, or for other purposes. Examples of techniques of use for nucleic acid amplification are found in Berger, Sambrook, and Ausubel, as well as Mullis et al., U.S. Pat. No. 4,683,202 (1987); and, PCR Protocols A Guide to Methods and Applications, Innis et al., Eds., Academic Press Inc., San Diego, Calif. (1990). PCR-based screening methods have been disclosed. [See, e.g., Wilfinger et al. BioTechniques, 22(3): 481-486 (1997).]

Nucleic Acid Separation Methods

In some embodiments, it may be of use to separate the amplification product from the template and the excess primer for the purpose of determining whether specific amplification has occurred. In one embodiment, amplification products are separated by agarose, agarose-acrylamide or polyacrylamide gel electrophoresis using standard methods. (See Sambrook et al., 1989). Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

Detection Methods

Products may be visualized in order to confirm amplification of the sequences of interest, such as disease associated NALP1 genetic variations. One typical visualization method involves staining of a gel with ethidium bromide and visualization under UV light. Alternatively, if the amplification products are labeled with radio- or fluorometrically-labeled nucleotides, the amplification products can then be exposed to X-ray film or visualized under the appropriate stimulating spectra, following separation.

In some embodiments, visualization is achieved indirectly. In accordance with these embodiments, after separation of amplification products, a labeled nucleic acid probe such as a probe is brought into contact with the amplified marker sequence. In certain embodiments, the probe can include an oligonucleotide capable of binding at least a portion of a nucleic acid sequence of the NALP1 gene, NALP1 promoter region or combination thereof including but not limited to, one or more genetic variations associated with vitiligo or vitiligo-associated autoimmune/autoinflammatory disease (VAAAD) selected from the group consisting of rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO:1)), rs2670660/C, rs2733359/G, rs35658367/ATGA, rs2716914/C, rs878329/G, rs7223628/G, rs8182352/G, rs4790796/A, rs4790797/T, rs8182354/A, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’ haplotype; any SNP listed in Table 9; any two-SNP haplotype as listed in Table 11 or the reverse complement of any of these variations, and a combination thereof.

In certain particular embodiments, the probe can be conjugated to a detectible agent such as a chromophore but may be radiolabeled. In another embodiment, the probe is conjugated to a binding partner, such as an antibody or biotin, and the other member of the binding pair carries a detectable moiety. In one embodiment, detection is by a labeled probe. The techniques involved are well known to those of skill in the art and can be found in many standard books on molecular protocols. (See Sambrook et al, 1989) For example, chromophore or radiolabel probes or primers identify the target during or following amplification.

In addition, amplification products described herein may be subjected to sequence analysis to identify specific kinds of variations using standard sequence analysis techniques. General techniques for determination of the DNA sequence of amplification products are well known in the art and include standard dideoxy sequencing by the Sanger technique.

### Covalent Modification of Nucleic Acids

A variety of cross-linking agents, alkylating agents and radical generating species may be used to bind, label, crosslink, detect, and/or cleave nucleic acids. For example, methods known in the art disclose covalent bonding of a single-stranded DNA fragment with alkylating derivatives of nucleotides complementary to target sequences. Other techniques known in the art concern sequence-specific cleavage of single-stranded DNA mediated by incorporation of a modified nucleotide which was capable of activating cleavage. Other methods can include covalent crosslinking to a target nucleotide using an alkylating agent complementary to the single-stranded target nucleotide sequence. A photoactivated crosslinking to single-stranded oligonucleotides mediated by psoralen was can be used.

Labeling Moieties

In various embodiments, nucleic acids, proteins, antibodies, aptamers and other molecules of use for detecting the presence or absence of specific target sequences, such as disease-associated genetic variations in NALP1 or the NALP1 promoter region, may be labeled using one or more moieties. Molecules may be labeled with photodetectable labels, radioisotope labels, enzymatic labels, chemiluminescent labels, PET (positron emission tomography) or NMR (nuclear magnetic resonance labels), or any other detectable labels known in the art. In one embodiment, detection molecules may be labeled with photodetectable labels. Exemplary photodetectable labels of use may include, but are not limited to, Alexa 350, Alexa 430, AMCA, aminoacridine, BODIPY 630/650, BODIPY 650/665, BODIPY-FL, BODIPY-R6G, BODIPY-TMR, BODIPY-TRX, 5-carboxy-4′,5′-dichloro-2′,7′-dimethoxy fluorescein, 5-carboxy-2′,4′,5′,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FAM, dansyl chloride, Fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin C, phycocyanin R, thiamine, phycoerythrocyanin, phycoerythrin R, REG, Rhodamine Green, rhodamine isothiocyanate, Rhodamine Red, ROX, TAMRA, TET, TRIT (tetramethyl rhodamine isothiol), Tetramethylrhodamine, and Texas Red.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging agents include astatine²¹¹, ¹⁴carbon, ⁵¹chromium, ³⁶chlorine, ⁵⁷cobalt, ⁵⁸cobalt, copper⁶², copper⁶⁴, copper⁶⁷, ¹⁵²Eu, fluorine¹⁸, gallium⁶⁷, gallium⁶⁸, ³hydrogen, iodine¹²³, iodine¹²⁴, iodine¹²⁵, iodine¹³¹, indium¹¹¹, ⁵²iron, ⁵⁹iron, ³²phosphorus, ³³phosphorus, rhenium¹⁸⁶, rhenium¹⁸⁸, Sc⁴⁷, ⁷⁵selenium, silver¹¹¹, ³⁵sulphur, technicium^{94m} technicium^{99m} yttrium⁸⁶ and yttrium⁹⁰. ¹²⁵I is often being preferred for use in certain embodiments, and technicium^{99m} and indium¹¹¹ are also often preferred due to their low energy and suitability for long range detection.

### Aptamers

In certain embodiments, aptamers against at least a portion or NALP1 protein or nucleic acid such as mRNA or cDNA may be of use for detecting NALP1 variants or potentially for inhibiting NALP1 activity as part of a therapeutic treatment for autoimmune/autoinflammatory disease. Methods of constructing and determining the binding characteristics of aptamers are well known in the art. For example, such techniques are described in U.S. Pat. Nos. 5,582,981, 5,595,877 and 5,637,459, each incorporated herein by reference.

Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding.

Aptamers need to contain the sequence that confers binding specificity, but may be extended with flanking regions and otherwise derivatized. In preferred embodiments, the NALP1 binding sequences of aptamers may be flanked by primer-binding sequences, facilitating the amplification of the aptamers by PCR or other amplification techniques. In a further embodiment, the flanking sequence may comprise a specific sequence that preferentially recognizes or binds a moiety to enhance the immobilization of the aptamer to a substrate.

Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

The aptamers used as starting materials to determine specific binding sequences may be single-stranded or double-stranded DNA or RNA. In a preferred embodiment, the sequences are single-stranded DNA, which is less susceptible to nuclease degradation than RNA. In preferred embodiments, the starting aptamer will contain a randomized sequence portion, generally including from about 10 to 400 nucleotides, more preferably 20 to 100 nucleotides. The randomized sequence is flanked by primer sequences that permit the amplification of aptamers found to bind to the target. For synthesis of the randomized regions, mixtures of nucleotides at the positions where randomization is desired may be added during synthesis.

Methods for preparation and screening of aptamers that bind to particular targets of interest are well known, for example U.S. Pat. No. 5,475,096 and U.S. Pat. No. 5,270,163, each incorporated by reference. The technique generally involves selection from a mixture of candidate aptamers and step-wise iterations of binding, separation of bound from unbound aptamers and amplification. Because only a small number of sequences (possibly only one molecule of aptamer) corresponding to the highest affinity aptamers exist in the mixture, it is generally desirable to set the partitioning criteria so that a significant amount of aptamers in the mixture (approximately 5-50%) are retained during separation. Each cycle results in an enrichment of aptamers with high affinity for the target. Repetition for between three to six selection and amplification cycles may be used to generate aptamers that bind with high affinity and specificity to the target, such as NALP1, caspases, ASC, interleukins, or their receptors.

### Phage Display

Since a number of known caspase inhibitors are short peptides, phage display may be well suited to identify novel caspase, NALP1, ASC, interleukin, or interleukin receptor inhibitors. Such inhibitory peptides may be identified by any method known in the art, including but not limiting to the phage display technique. Various methods of phage display and techniques for producing diverse populations of peptides are well known in the art. For example, U.S. Pat. Nos. 5,223,409; 5,622,699 and 6,068,829, disclose methods for preparing a phage library. The phage display technique involves genetically manipulating bacteriophage so that small peptides can be expressed on their surface.

Peptide amino acid sequences that bind to a given target molecule, such as NALP1, caspases, ASC, interleukins, or their receptors, may be isolated by panning (Pasqualini and Ruoslahti, 1996, Nature 380:364-366; Pasqualini, 1999, The Quart. J. Nucl. Med. 43:159-162). In certain embodiments, the phage may be propagated in host bacteria between rounds of panning. Rather than being lysed by the phage, the bacteria may instead secrete multiple copies of phage that display a particular insert. If desired, the amplified phage may be exposed to the target molecule again and collected for additional rounds of panning. Multiple rounds of panning may be performed until a population of selective or specific binders is obtained.

### Antibodies

In some embodiments, diagnostic assays for NALP1 protein or NALP1 nucleic acid may be of use to identify the presence of genetic variations predisposing to autoinflammatory/autoimmune disease. For example, where such variations result in an increase or decrease in NALP1 protein production, which in turn promotes autoimmune/autoinflammatory disease, then antibody based assays may be of use to indirectly detect the underlying genetic variation. Alternatively, in cases where NALP1 genetic variations located in the coding region of the gene produce changes in the amino acid sequence of the encoded proteins, sequence specific antibodies that differentiate the mutant protein from normal protein may be used.

The term “antibody” is used herein to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlowe and Lane, 1988, Antibodies: A Laboratory Manual, Cold Spring Harbor Laboratory).

Polyclonal Antibodies

Polyclonal antibodies may be prepared by immunizing an animal with an immunogen and collecting antisera from that immunized animal or any means known in the art. A wide range of animal species may be used for the production of antisera. Typically an animal used for production of antisera is a non-human animal, for example, rabbits, mice, rats, hamsters, pigs or horses. Because of the relatively large blood volume of rabbits, a rabbit is a preferred choice for production of polyclonal antibodies.

Antibodies, both polyclonal and monoclonal, may be prepared using conventional immunization techniques. A composition containing antigenic epitopes may be used to immunize one or more experimental animals, such as a rabbit or mouse, which will then proceed to produce specific antibodies. Polyclonal antisera may be obtained, after allowing time for antibody generation, simply by bleeding the animal and preparing serum samples from the whole blood.

In certain embodiments, a given composition may vary in its immunogenicity. Therefore a boost in the host immune system may be needed, as may be achieved by coupling an immunogen to a carrier. Exemplary and preferred carriers are keyhole limpet hemocyanin (KLH) and bovine serum albumin (BSA). Other albumins such as ovalbumin, mouse serum albumin or rabbit serum albumin also may be used as carriers. Means for conjugating an antigen to a carrier protein are well known and include cross-linkers such as glutaraldehyde, m-maleimidobenzoyl-N-hydroxysuccinimide ester, carbodiimide and bis-biazotized benzidine.

The amount of immunogen composition used in the production of polyclonal antibodies varies upon the nature of the immunogen as well as the animal used for immunization. A variety of routes may be used to administer the immunogen (subcutaneous, intramuscular, intradermal, intravenous and intraperitoneal). The production of polyclonal antibodies may be monitored by sampling blood of the immunized animal at various points following immunization. A second, booster, injection also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal Antibodies

Monoclonal antibodies may be readily prepared through use of well-known techniques, such as those exemplified in U.S. Pat. No. 4,196,265. Typically, this technique involves immunizing a suitable animal with a selected immunogen composition. The immunizing composition is administered in a manner effective to stimulate antibody producing cells. Cells from rodents such as mice and rats are preferred. Mice are more preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B-lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus, and those using polyethylene glycol (PEG), such as 37% (v/v) PEG, have been described. The use of electrically induced fusion methods is also appropriate. Methods for selecting and culturing of hybridomas can be performed by means known in the art. Assays for selecting hybridomas contemplated herein are sensitive, simple and rapid, for example radioimmunoassays, enzyme immunoassays, cytotoxicity assays, plaque assays, dot immunobinding assays, and the like.

Then selected hybridomas can be further used by means known in the art for generation and isolation of monoclonal antibodies. mAbs produced by either means may be further purified, if desired, using filtration, centrifugation, and various chromatographic methods such as HPLC or affinity chromatography.

Production of Antibody Fragments

Some embodiments of the claimed methods and/or compositions may concern antibody fragments. Exemplary methods for producing antibody fragments are disclosed in U.S. Pat. No. 4,036,945; U.S. Pat. No. 4,331,647; Nisonoff et al., 1960, Arch. Biochem. Biophys., 89:230; and Porter, 1959, Biochem. J., 73:119.

Other methods of cleaving antibodies, such as separation of heavy chains to form monovalent light-heavy chain fragments, further cleavage of fragments or other enzymatic, chemical or genetic techniques also may be used, so long as the fragments bind to the antigen that is recognized by the intact antibody. Alternatively, the variable chains may be linked by an intermolecular disulfide bond or cross-linked by chemicals such as glutaraldehyde. See Sandhu, 1992, Crit. Rev. Biotech., 12:437.

Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains, connected by an oligonucleotides linker sequence. Another contemplated form of an antibody fragment is a peptide coding for a single complementarity-determining region (CDR). In various embodiments, antibody fragments may be of use as inhibitors of NALP1, caspases, ASC, interleukins, or their receptors.

Chimeric and Humanized Antibodies

Where antibodies or antibody fragments are administered in vivo in human subjects, it may be preferred to provide chimeric or humanized antibodies to reduce the host immune response to the foreign antibody. A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. Methods for constructing chimeric antibodies are well known in the art (e.g., Leung et al., 1994, Hybridoma 13:469).

A chimeric monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. To preserve the stability and antigen specificity of the humanized monoclonal, one or more human FR residues may be replaced by the mouse counterpart residues. Humanized monoclonal antibodies may be used for therapeutic treatment of subjects. The affinity of humanized antibodies for a target may also be increased by selected modification of the CDR sequences (WO0029584A1). Techniques for production of humanized monoclonal antibodies are well known in the art. (See, e.g., Jones et al., 1986, Nature, 321:522; Riechmann et al., Nature, 1988, 332:323; Verhoeyen et al., 1988, Science, 239:1534; Carter et al., 1992, Proc. Nat'l Acad. Sci. USA, 89:4285; Sandhu, Crit. Rev. Biotech., 1992, 12:437; Tempest et al., 1991, Biotechnology 9:266; Singer et al., J. Immun., 1993, 150:2844.)

In another embodiment, an antibody may be a human monoclonal antibody. Such antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994).

Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Phamacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique, as discussed above, may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In another alternative, transgenic animals that have been genetically engineered to produce human antibodies may be used to generate antibodies against essentially any immunogenic target, using standard immunization protocols as discussed above. A non-limiting example of such a system is the XenoMouse® (e.g., Green et al., 1999, J. Immunol. Methods 231:11-23, incorporated herein by reference) from Abgenix (Fremont, Calif.). In the XenoMouse® and similar animals, the mouse antibody genes have been inactivated and replaced by functional human antibody genes, while the remainder of the mouse immune system remains intact.

### Immunodetection

Antibodies may be used for quantitation of NALP1 protein by any of a variety of known immunodetection methods, for example by ELISA assay. Anti-NALP1 antibodies may be immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera, such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation. Following formation of specific immunocomplexes between the test sample and the bound antibody, and subsequent washing, the occurrence and even amount of immunocomplex formation may be determined by subjecting the same to a second antibody having specificity for NALP1 that differs from that of the first antibody.

Appropriate conditions preferably include diluting the sample with diluents such as BSA, bovine gamma globulin (BGG), and phosphate buffered saline (PBS)/Tween®. These added agents also tend to assist in the reduction of nonspecific background. The layered antisera is then allowed to incubate for from about 2 to about 4 h, at temperatures preferably on the order of about 25° to about 27° C. Following incubation, the antisera-contacted surface is washed so as to remove non-immunocomplexed material. A preferred washing procedure includes washing with a solution such as PBS/Tween® or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-IgG for a period of time and under conditions which favor the development of immunocomplex formation (e.g., incubation for 2 h at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The antibody compositions may also be of use in immunoblot, Western blot or any other known type of immunodetection analysis. Immunologically-based detection methods for use in conjunction with Western blotting include enzymatically-, radiolabel-, or fluorescently-tagged secondary antibodies against NALP1 protein or the primary antibody.

### Preparation of Immunoconjugates

In certain embodiments, immunoconjugates may be used to target one or more genetic variations herein. In one example, immunoconjugated contemplated herein may be prepared by known methods of linking antibodies with lipids, carbohydrates, protein, or other atoms and molecules. For example, the binding molecules described herein can be conjugated with one or more of the carriers described herein (e.g., lipids, polymers, liposomes, micelles, or nanoparticles) to form an immunoconjugate, and the immunoconjugate can incorporate a therapeutic or diagnostic agent either covalently, non-covalently, or otherwise. Further, any of the binding molecules described herein can be conjugated with one or more therapeutic or diagnostic agents described herein, or additional carriers. Generally, one therapeutic or diagnostic agent may be attached to each binding molecule but more than one therapeutic agent or diagnostic agent can be attached to the same binding molecule. In one embodiment, the antibody fusion proteins contemplated herein may include two or more antibodies or fragments thereof and each of the antibodies that include this fusion protein may be conjugated with one or more of the carriers described herein. Additionally, one or more of the antibodies of the antibody fusion protein may have one or more therapeutic of diagnostic agent attached. Further, the therapeutic do not need to be the same but can be different therapeutic agents. For example, the compositions described herein may include a drug and a radioisotope.

In another embodiment, a carrier, therapeutic agent, or diagnostic agent may be attached to an immunoconjugate. As an alternative, peptides can be attached to an antibody component using a heterobifunctional cross-linker, such as N-succinyl 3-(2-pyridyldithio)proprionate (SPDP). General techniques for such conjugation are well known in the art. Alternatively, the carrier, therapeutic agent, or diagnostic agent can be conjugated via a carbohydrate moiety in the Fc region of an antibody.

### Carriers (Lipids, Liposomes, Micelles, Polymers, and Nanoparticles)

Any methods for formation of liposomes and micelles may be use and are known in the art. Nanoparticles or nanocapsules formed from polymers, silica, or metals, which are contemplated herein for drug delivery or imaging, have been described.

### Pharmaceutically Acceptable Excipients

In one embodiment, the immunoconjugates or compositions may include one or more pharmaceutically suitable excipients, one or more additional ingredients, or combination thereof. In another embodiment, the immunoconjugate or compositions disclosed herein may be formulated according to known methods to prepare pharmaceutically useful compositions, whereby the immunoconjugate or compositions are combined in a mixture with a pharmaceutically suitable excipient. Sterile phosphate-buffered saline is one example of a pharmaceutically suitable excipient. Other suitable excipients are well known to those in the art.

In another embodiment, the immunoconjugate or compositions disclosed herein can be formulated for intravenous administration via, for example, bolus injection or continuous infusion.

Formulations for injection may be presented for example in unit dosage form, e.g., in ampules or in multi-dose containers, with an added preservative. The compositions may take such forms as suspensions, solutions or emulsions in oily or aqueous vehicles, and can contain formulatory agents such as suspending, stabilizing and/or dispersing agents. Alternatively, the active ingredient may be in powder form for constitution with a suitable vehicle, e.g., sterile pyrogen-free water, before use. Additional pharmaceutical methods may be employed to control the duration of action of the therapeutic, diagnostic conjugate or naked antibody. For example, control release preparations may be prepared through the use of polymers to complex or adsorb the immunoconjugate or naked antibody. In one example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). Some of the rates of release of an immunoconjugate or antibody from such a matrix depends upon the molecular weight of the immunoconjugate or antibody, the amount of immunoconjugate, antibody within the matrix, and the size of dispersed particles. Saltzman et al., Biophys. J. 55: 163 (1989); Sherwood et al., supra. Other solid dosage forms are described in Ansel et al., Pharmaceutical Dosage Forms and Drug Delivery Systems, 5th Edition (Lea & Febiger 1990), and Gennaro (ed.), Remington's Pharmaceutical Sciences, 18th Edition (Mack Publishing Company 1990), and revised editions thereof.

In one embodiment, the immunoconjugate or compositions may also be administered to a mammal subcutaneously or even by other parenteral routes. Moreover, the administration may be by continuous infusion or by single or multiple boluses. In one example, the dosage of an administered immunoconjugate, fusion protein or naked antibody for humans will vary depending upon such factors as the patient's age, weight, height, sex, general medical condition and previous medical history etc. In a particular embodiment, the recipient may be administered a dosage of immunoconjugate or composition including the immunoconjugate that is in the range of from about 1 mg/kg to mg/kg as a single intravenous infusion, although a lower or higher dosage also may be administered as circumstances dictate. This dosage may be repeated as needed, for example, once per week for 4-10 weeks, preferably once per week for 8 weeks, and more preferably, once per week for 4 weeks. It may also be given less frequently, such as every other week for several months. The dosage may be given through various parenteral routes, with appropriate adjustment of the dose and schedule.

In one embodiment, for purposes of therapy, an immunoconjugate, or composition including the immunoconjugate, is administered to a subject in a therapeutically effective amount. A suitable subject for the therapeutic and diagnostic methods disclosed herein may be a human, although a non-human animal subject is also contemplated. For example, large and small domesticated animals, as well as, undomesticated animals and birds.

In one example an antibody preparation is said to be administered in a “therapeutically effective amount” if the amount administered is physiologically significant. An agent is physiologically significant if its presence results in a detectable change in the physiology of a recipient mammal. In particular, an antibody preparation is physiologically significant if its presence invokes an antitumor response or mitigates the signs and symptoms of an autoimmune disease state. A physiologically significant effect could also be the evocation of a humoral and/or cellular immune response in the recipient subject.

A drug can be included in the pharmaceutical composition in an amount sufficient to produce the desired effect upon the patient. The pharmaceutical compositions of the present invention can further comprise other chemical components, such as diluents and excipients. A “diluent” is a chemical compound diluted in a solvent, preferably an aqueous solvent, that facilitates dissolution of the drug in the solvent, and it may also serve to stabilize the biologically active form of the drug or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer-salts can control the pH of a solution at low concentrations, a buffered diluent rarely modifies the biological activity of a biologically active peptide.

In one embodiment herein, pharmaceutical compositions facilitate administration of humanized antibodies to an organism, preferably an animal, preferably a mammal. Particular mammals include bovine, canine, equine, feline, ovine, and porcine animals, and non-human primates. Humans are particularly preferred. Multiple techniques of administering or delivering a compound exist in the art include but are not limited to, oral, rectal (e.g., an enema or suppository) aerosol (e.g., for nasal or pulmonary delivery), parenteral (e.g., i.v., i.m., s.c.), and topical administration. Preferably, sufficient quantities of the composition or compound may be delivered to achieve the intended effect. The particular amount of composition or compound to be delivered will depend on many factors, including the effect to be achieved, the type of organism to which the composition is delivered, delivery route, dosage regimen, and the age, health, and sex of the organism. As such, the particular dosage of a composition or compound of embodiment disclosed herein in a given formulation is left to the ordinarily skilled artisan's discretion (e.g. the health provider's discretion).

Those skilled in the art will appreciate that when the pharmaceutical compositions of the present invention are administered as agents to achieve a particular desired biological result, which may include a therapeutic or protective effect(s) (including vaccination), it may be necessary to combine the composition or compounds disclosed herein with a suitable pharmaceutical carrier. The choice of pharmaceutical carrier and the preparation of the composition or compound as a therapeutic or protective agent will depend on the intended use and mode of administration. Suitable formulations and methods of administration of therapeutic agents include, but are not limited to, those for oral, pulmonary, nasal, buccal, ocular, dermal, rectal, or vaginal delivery.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, aerosol, droplet, or spray. Pills, tablets, suppositories, aerosols, powders, droplets, and sprays may have complex, multilayer structures and have a large range of sizes. Aerosols, powders, droplets, and sprays may range from small (approximately 1 micron) to large (approximately 200 micron) in size.

Pharmaceutical compositions disclosed herein may be used in the form of a solid, a lyophilized powder, a solution, an emulsion, a dispersion, a micelle, a liposome, and the like, wherein the resulting composition contains one or more of the targetable constructs or complexes of embodiments of the present invention, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Examples of a stabilizing dry agent includes triulose, preferably at concentrations of 0.1% or greater (See, e.g., U.S. Pat. No. 5,314,695).

Although individual needs may vary, determination of optimal ranges for effective amounts of pharmaceutical compositions is within the skill of the art. Human doses may be extrapolated from animal studies. Generally, the dosage required to provide an effective amount of a pharmaceutical composition, which may be adjusted by one skilled in the art, will vary depending on the age, health, physical condition, weight, type and extent of the disease or disorder of the recipient, frequency of treatment, the nature of concurrent therapy (if any) and the nature and scope of the desired effect(s).

Dosing of therapeutic compositions is dependent on severity and responsiveness of the disease state to be treated, with the course of treatment lasting from several days to several months, or until a cure is effected or a diminution of the disease state or symptom(s) is achieved. Optimal dosing schedules can be calculated from measurements of drug accumulation in the body of the patient. The term “patient” is intended to encompass animals (e.g., cats, dogs and horses) as well as humans. Persons of ordinary skill can easily determine optimum dosages, dosing methodologies and repetition rates. Optimum dosages may vary depending on the relative potency of individual therapeutic agents.

The range of doses is broad, since in general the efficacy of a therapeutic effect for different mammals varies widely with doses typically being 20, 30 or even 40 times smaller (per unit body weight) in man than in the rat. In general, dosage is from 01 μg to 100 mg per kg of body weight, preferably 01 μg to 10 mg/kg of body weight, 01 μg to 50 μg/kg of body weight, 01 μg to 100 mg/kg of body weight, 01 μg to 10 mg/kg of body weight, 01 μg to 1 mg/kg of body weight, 01 μg to 100 μg/kg of body weight, 01 μg to to 10 μg/kg of body weight, 01 μg to 1 μg/kg of body weight, 01 μg to 10 μg/kg of body weight, 01 μg to 1 μg/kg of body weight, 01 μg to 0.1 μg/kg of body weight, and ranges based on the boundaries of the preceding ranges of concentrations. Thus, for example, the preceding description of dosages encompasses dosages within the range of 10 mg to 100 mg per kg of body weight, 1.0 mg to 100 mg/kg of body weight, 0.1 mg to 100 mg/kg of body weight, etc.

In one embodiment, doses may be given once or more daily, weekly, monthly or yearly, or even once every 2 to 5 or more years. Persons of ordinary skill in the art may estimate repetition rates for dosing based on measured residence times and concentrations of the targetable construct or complex in bodily fluids or tissues. Following successful treatment, it may be desirable to have the patient undergo maintenance therapy to prevent the recurrence of the disease state or risk of recurrence, wherein the therapeutic agent is administered in maintenance doses, ranging from 0.01 .mu.g to 100 mg per kg of body weight, once or more daily, to once every 5 years.

In one embodiment, a particular dose may be calculated according to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data.

In one embodiment, an individual patient's dosage may be adjusted as the progress of the disease is monitored. Blood levels of the composition or agent used in a patient may be measured to see if the dosage needs to be adjusted to attain desired results.

In one example, pharmacogenomics may be used to determine which compositions, agents ordosages thereof, are most likely to be effective for a given individual.

### Kit Components

All the materials and reagents required for detecting, measuring, or sequencing NALP1 and variants thereof, for example detection of one or more SNPs, haplotypes or combination thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally can include but are not limited to, a suitable container means, distinct containers for each individual reagent and enzyme, as well as for each primer or probe. Alternatively, where antibody based assays are contemplated, the antibodies, control peptides, secondary antibodies, reagents and/or any other materials may also be provided in kit form. The materials and reagents may be packaged in individual containers within a box or other package.

Some embodiments concern a kit including a container means and at least one probe capable of binding rs6502867 of the NALP1 gene. In other embodiments a kit may include at least one probe capable of binding rs6502867 of the NALP1 gene. and one or more additional probes capable of binding to rs12150220, rs2670660, rs878329, rs7223628, rs8182352, rs4790796, or rs47907970.

### Methods for Screening Active Compounds

In Vitro Assays

Certain embodiments involving identification of inhibitors of NALP1 or other proteins may include the screening of compounds that bind to NALP1 or a fragment thereof. The polypeptide or fragment may be either free in solution, fixed to a support, or expressed in or on the surface of a cell. Either the polypeptide or the compound may be labeled, thereby permitting the determination of binding.

In another embodiment, the assay may measure the inhibition of binding of NALP1 to a natural or artificial substrate or binding partner. Competitive binding assays can be performed in which one of the agents (NALP1, binding partner or compound) is labeled. Usually, the polypeptide will be the labeled species. One may measure the amount of free label versus bound label to determine binding or inhibition of binding.

Another technique for high throughput screening of compounds is described in WO 84/03564, the contents of which are incorporated herein by reference. Large numbers of small peptide test compounds are synthesized on a solid substrate. The peptide test compounds are reacted with NALP1 and washed. Bound polypeptide is detected by various methods.

Purified NALP1 can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the NALP1 active region to a solid phase.

### In Vivo Assays

Other embodiments may encompass the use of various animal models. By developing or isolating NALP1 genetic variants and inserting them into transgenic cells or model animals, such as nude mice, it may be possible to produce animal model systems for autoinflammatory/autoimmune disease, which may be used to screen for therapeutic agents. Where desired, knock-out techniques known in the art may be utilized to inhibit expression of endogenous NALP1 genes or their analogs.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of the symptoms of autoimmune/autoinflammatory disease, improvement in immune system function, assays for various cytokines, subtypes of T or B cells or other immune system regulatory or effector cells and improved food intake.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents

### EXAMPLES

The following examples are included to illustrate various embodiments of the invention. It should be appreciated by those of skill in the art that the techniques disclosed in the examples which follow represent techniques discovered to function well in the practice of the claimed methods. However, those of skill in the art should, in light of the present disclosure, appreciate that many changes may be made in the specific embodiments which are disclosed and still obtain a like or similar result without departing from the spirit and scope of the invention.

### Example 1

In one exemplary study, 177 single-nucleotide polymorphisms (SNPs) spanning the 17p13 linkage peak were tested for association with disease. A strong candidate gene was identified. DNA in and around the gene was sequenced to identify additional SNPs. Then, a second round of tests was performed for association using some of the 177 SNPs, thus elucidating the association with disease in the gene and its extended promoter region in fine detail. A candidate gene was identified as NALP, encoding NACHT leucine-rich-repeat protein 1, a regulator of the innate immune system.

In another exemplary method, fine-scale association mapping with the use of DNA from affected families and additional SNPs in and around NALP1 showed an association of specific variants with vitiligo, an extended autoimmune and autoinflammatory disease phenotype, or a combination of the diseases. Conditional logistic-regression analysis of NALP1 SNPs indicated that at least two variants contribute independently to the risk of disease.

### Methods

In one exemplary method, 656 subjects from 114 USA and UK Caucasian extended families with multiple autoimmune/autoinflammatory disease, each including at least two members with generalized vitiligo and at least one with one or more vitiligo-associated autoimmune/autoinflammatory disease (autoimmune thyroid disease, rheumatoid arthritis, adult-onset autoimmune diabetes mellitus, psoriasis, pernicious anemia, systemic lupus erythematosus, and Addison's disease; Table 1). All available affected and unaffected family members filled out a detailed questionnaire providing clinical history of about 50 autoimmune/autoinflammatory diseases, including Addison's disease, allergies/asthma/eczema, ankylosing spondylitis (Strümpell-Marie disease/Bekhterev-Strümpel syndrome/spondyloarthritis), antiphospholipid syndrome (anti-cardiolipin syndrome), APECED syndrome (autoimmune polyendocrine syndrome type 1; APS1), autoimmune hemolytic anemia, autoimmune hepatitis (non-infectious chronic active hepatitis), Behçet's disease, bullous pemphigoid, cardiomyopathy, celiac disease (celiac sprue/gluten enteropathy), chronic inflammatory demyelinating polyneuropathy, Churg-Strass syndrome (allergic granulomatosis), cicatrical pemphigoid (mucous membrane pemphigoid/benign pemphigoid), CREST syndrome, cold agglutinin disease, diabetes (specifying age of onset and type, including insulin-dependent diabetes mellitus/type 1/juvenile diabetes, non-insulin-dependent diabetes mellitus/type 2/adult-onset, unknown, and type of treatment (pills/diet/insulin/none), essential mixed cryoglobulinemia, fibromyalgia-fibromyositis syndrome, Guillain-Barré syndrome, IgA nephropathy, idiopathic thrombocytic purpura (ITP), inflammatory bowel disease (Crohn's disease/ulcerative colitis/irritable bowel syndrome), hypo- or hyper-parathyroidism, kidney disease (glomerulonephritis/nephrosis/nephritic syndrome), juvenile arthritis, lichen planus, multiple sclerosis, myasthenia gravis, pernicious anemia, pemphigus vulgaris, polyarteritis nodosa, polychondritis, polymyalgia rheumatica, polymyositis and dermatomyositis (PM_DM syndrome), primary biliary cirrhosis, psoriasis, Raynaud's phenomenon, Reiter's syndrome, rheumatic fever, rheumatoid arthritis, sarcoidosis, Schmidt syndrome (autoimmune polyendocrine syndrome type 2; APS2), Sjogren's syndrome, Stiff-man syndrome (Moersch-Woltmann syndrome), systemic lupus erythematosus (lupus; SLE), Takayasu arteritis, temporal arteritis (giant cell arteritis), thyroid disease (Graves disease/myxedema/hyperthyroidism, Hashimoto's thyroiditis/goiter/hypothyroidism), uveitis, and vitiligo. In some cases specific diagnoses were checked by review of clinical records and clinical tests; all data were reviewed by the study investigators, and most family members (affected and unaffected) were personally examined by one of the study staff. Diagnoses that were considered questionable by standard diagnostic criteria were excluded. All patients with vitiligo provided a skin-lesion map.

Family Member Criteria: All data from the questionnaires and the lesion maps and the study staff examined most family members, both affected and unaffected. Inclusion criteria for family members with generalized vitiligo were the presence of depigmented patches of skin that were acquired; that had changed in extent and boundaries over time; that were nonfocal and bilateral; and that typically initially involved the fingers, hands, feet, face, or crural areas; and that were not associated with concurrent underlying eczema or psoriasis or with exposure to depigmenting chemicals. Family members with diagnoses that were considered to be questionable on the basis of standard diagnostic criteria were excluded. A summary of autoimmune diseases in the 114 study families was generated (Table 1).

### Genotyping

In other exemplary methods, DNA was prepared from peripheral-blood specimens with the use of a genomic DNA purification kit (Puregene, Gentra Systems) or from saliva specimens with the use of a DNA self-collection kit (Oragene, DNA Genotek). In one example, the Illumina genotyping service was used to genotype family members in the first series, assaying 177 known single-nucleotide polymorphisms (SNPs) selected from the Illumina SNP Knowledge Resource. Each SNP had a minor allele frequency exceeding 0.10 (i.e., the less common variant of the SNP occurred on at least 10% of chromosomes) in whites; altogether, these SNPs captured approximately 18% of the common genetic variation (r2≧0.5) across the genetic-linkage region of chromosome 17p (approximately 11.3 cM, or 6.19 Mb). In another example, family members were genotyped in the second series for the 23 SNPs that were significantly associated with disease in the first series according to both the pedigree disequilibrium test 14 and family-based association test. The two insertion-deletion polymorphisms and 78 additional SNPs were genotyped in all 114 families (both series combined). Most of these 78 SNPs were identified by sequencing NALP1, the gene for NACHT leucine-rich-repeat protein 1, and its extended promoter region in 15 genetically informative family members.

### DNA Sequencing

In another exemplary method, after narrowing the 17p13 autoimmunity locus to NALP1 and its extended promoter region, DNA sequence variants within this region were identified that could be used for further tests of association and for identification of the causal SNP or SNPs. Therefore, a 82.9 kb of the NALP1 gene and its extended promoter region were sequenced in each of four parents (two from each series) who were heterozygous for a haplotype (a set of SNPs that occur on a single chromosome) of three adjacent Illumina SNPs (rs3926687, rs2733359, and rs878329) that initially appeared to confer a high risk (haplotype 1) and who had transmitted this haplotype to at least one affected offspring. The same region in 11 unrelated patients was sequenced who were homozygous for haplotype 1 (8 from the first series and 3 from the second series). Most of these 15 patients had vitiligo and at least one other autoimmune disease. The sequenced regions included a contiguous segment of 69.1 kb that spanned the extended NALP1 promoter region and exons 1, 2, and 3, as well as 11 individual segments containing exons 4 through 18 with 90 to 500 bp of adjacent intronic sequences. Several small introns were sequenced completely. The regions sequenced define the five known alternatively spliced isoforms of NALP1 messenger RNA (mRNA); approximately 77% of the sequence was determined by analyzing both DNA strands. Nucleotide positions were obtained from the human genome sequence for chromosome 17 from the National Center for Biotechnology Information (NCBI) (Build 36).

In another example, the predicted effect of the substitution of histidine for leucine at position 155 (Leu155→His) on the secondary structure of the NALP1 protein was assessed with the use of the protein structure prediction server (PSIPRED). The potential effects of both alleles of all promoter region variants on transcription-factor binding motifs were predicted with the use of the Transcription Element Search System (TESS) 17 and rVista 2.0 software.18

### Statistical Analysis

In one exemplary method, details on preliminary analyses, genetic-linkage analyses, and conditional logistic-regression analysis were performed (data not shown). Hardy-Weinberg equilibrium was tested for in founders (the earliest specified persons in lineages) and in persons not in the lineage, such as spouses, in all 114 families. Calculations of linkage disequilibrium between markers of the NALP1 region were carried out with for example, Haploview software, version 3.32. The association of each marker were calculated with vitiligo or with an expanded autoimmune phenotype considering as affected persons with any autoimmune or autoinflammatory disease associated with vitiligo using the family based association test, version 1.5.5, and the pedigree disequilibrium test, version 5.1, which provides a combined statistic accounting for allele transmission to both affected offspring and unaffected offspring. Haplotype-based transmission-disequilibrium statistics were calculated with the use of the family-based association test, version 1.5.5. P values of less than 0.05 were considered to indicate statistical significance.

### Genetic-Linkage Analysis

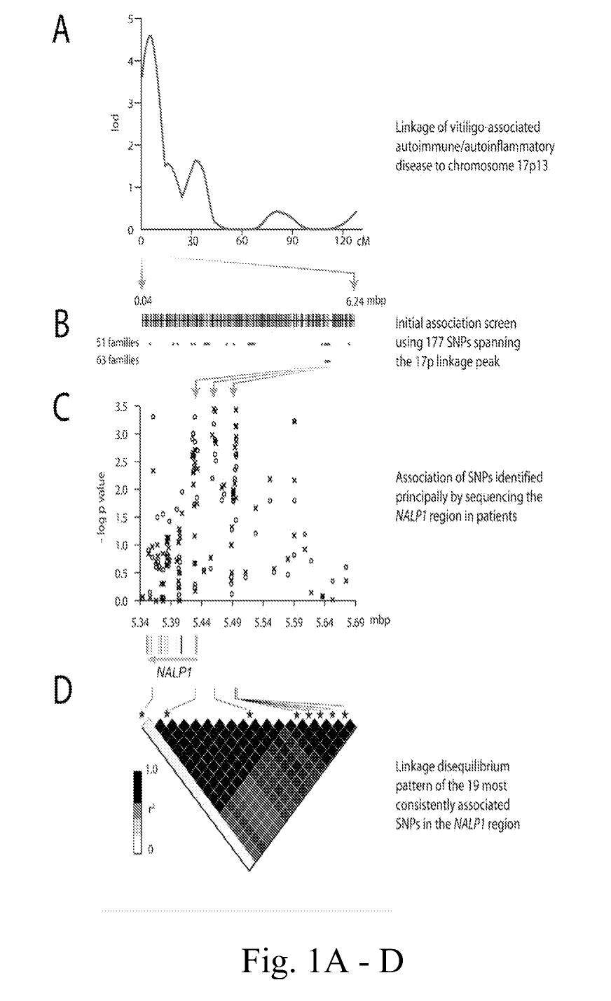

A genetic linkage between a locus on chromosome 17p13 and multiple autoimmune disease associated with vitiligo were previously reported. A result obtained by genotyping microsatellite markers across the genome in 51 extended families (the first series). In other families with vitiligo only, there was no linkage to this chromosomal region. With the use of these same data but an improved error-checking algorithm, the maximum multipoint lod score was 4.59 (2.14×10⁶)—an improvement on the previously obtained lod score of 4.00. The multipoint maximum lod score occurred at approximately 4.3 cM on chromosome 17p; the linkage region encompassing a 1-lod reduction from the maximum (thus, the region that probably contains the causal gene) spanned 11.3 cM (FIG. 1A).

### Family-Based Association Studies

There are approximately 80 known or predicted genes within the 11.3-cM region spanning the linkage peak on chromosome 17p. To determine which of these genes is most likely to contribute to multiple autoimmune disease associated with vitiligo, the families in the first series for the 177 SNPs spanning the linkage peak were genotyped. The frequencies of all SNPs in founders and spouses who had married into the family were consistent with Hardy-Weinberg equilibrium. Genetic-linkage analysis incorporating both SNP and microsatellite data did not result in a significant narrowing of the linkage peak (results not shown). Then association of these SNP genotypes were tested with vitiligo alone and with the entire group of autoimmune and autoinflammatory diseases associated with vitiligo, considering a family member with any of these diseases as “affected.” Both the pedigree disequilibrium test and the family-based association test were used which yielded generally similar results (data not shown). Both tests showed that the 23 SNPs were associated with the vitiligo phenotype, with the expanded autoimmune and autoinflammatory disease phenotype, or with both, including a cluster of five adjacent SNPs—rs2301582, rs9889625, rs3926687, rs2733359, and rs878329—spanning a 117-kb region (FIG. 1B).

In another exemplary method, to assess the reproducibility of these candidate association signals, an independent analysis was carried out in which the 23 significant SNPs were genotyped in a second series of 63 extended families with multiple autoimmune disease associated with vitiligo. The results of this analysis provided support for an association of three of the five adjacent SNPs—rs3926687, rs2733359, and rs878329—with the vitiligo phenotype and with the expanded autoimmune and autoinflammatory disease phenotype, both in the second series and in all 114 families ((FIG. 1B) other data not shown).

These three high-risk SNPs span a 61-kb segment that includes the proximal coding region and the extended promoter region of NALP1, which encodes a key regulator of the innate immune system (FIG. 1C). The genomic region around NALP1 is gene-sparse; SNPs located downstream of NALP1 were not associated with disease, and the closest upstream gene, KIAA0523, is more than 486 kb toward the centromere from NALP1. The result of the family-based association test showed that a preliminary haplotype defined by these three SNPs—rs3926687, rs2733359, rs878329—(haplotype 1) had the most significant association with both the vitiligo phenotype. The expanded autoimmune and autoinflammatory disease phenotype in the first series (P=0.01 and P=0.009, respectively), in the second series (P=0.03 and P=0.02, respectively), and in both series combined (P<0.001 for both comparisons; odds ratio for vitiligo, 1.85; 95% confidence interval [CI], 1.25 to 2.71; odds ratio for autoimmune and autoinflammatory disease, 1.79; 95% CI, 1.25 to 2.56). Of the 177 SNPs initially tested, 98 clustered into 21 blocks of linkage disequilibrium. With regard to correction for multiple testing, the 177 SNPs constituted approximately 100 independent tests (21 blocks of linkage disequilibrium and the 79 remaining SNPs), with a corrected P value of 0.04 for vitiligo-associated autoimmune and autoinflammatory diseases.

In other exemplary methods, sequence analysis of NALP1 and its extended promoter region was performed in 11 persons with vitiligo who were homozygous for haplotype 1 and in 4 unaffected heterozygotes who had transmitted haplotype 1 to at least one affected offspring. The analysis yielded a total of 261 sequence variants (Table 8), 54 of which were newly discovered. A segment of 524 bp was identified that was missing from the NCBI human chromosome 17 sequence, immediately after nucleotide 5,466,866. To define more precisely the NALP1 genomic region that confers susceptibility to autoimmune and autoinflammatory disease, we genotyped all 114 families for 78 additional SNPs (identified by means of sequence analysis (FIG. 1C) and two small insertion-deletion polymorphisms (data not shown), selected on the basis of their physical positions, HapMap tag-SNP status, minor allele frequencies, and potential functional significance. The genotype frequencies of all variants tested were consistent with Hardy-Weinberg equilibrium and were similar in the two series (data not shown). As shown in FIG. 1C, many NALP1 region variants were associated with vitiligo (with P values ranging from 0.048 to <0.001 and odds ratios ranging from 1.39 to 2.08) or with associated autoimmune and autoinflammatory disease (with P values ranging from 0.04 to <0.001 and odds ratios ranging from 1.25 to 1.93), in a pattern broadly distributed across the proximal portion of the NALP1 structural gene and its extended promoter region.

A stronger association was observed for the expanded autoimmune and autoinflammatory disease phenotype than for the smaller vitiligo subgroup (data not shown). Apparent associations of disease with multiple markers in the NALP1 region may reflect multiple independent causal variants or may be a consequence of linkage disequilibrium between multiple markers and one true causal variant. The alignment of the genomic positions (FIG. 1C) and the linkage-disequilibrium pattern (FIG. 1D) of the 19 NALP1 region markers (17 SNPs and 2 insertion-deletion polymorphisms) for which an association with disease was replicated in the two series (by means of the pedigree disequilibrium test and the family-based association test) suggested that at least two markers might be independently associated with disease. To distinguish markers that might reflect independent variants from those that merely reflect linkage disequilibrium, conditional logistic-regression analyses 21 of these 19 markers (data not shown). On the basis of this analysis, three markers (rs6502867, rs8182352, and rs4790797) had the largest individual effects both for the expanded autoimmune and autoinflammatory disease phenotype (odds ratio, 1.93, 1.81, and 1.82, respectively; P<0.001 for all three markers) and for the vitiligo phenotype (odds ratios, 2.08, 2.01, and 2.01, respectively; P<0.001 for all three markers). The inclusion of rs6502867 significantly improved the fit of logistic-regression models that also included any 1 of the 18 other markers; conversely, the fit of the model including rs6502867 was significantly improved by the inclusion of any 1 of 15 of the 18 other markers (Table 10). These results provide further support for the existence of at least two independent variants in the NALP1 region associated with the risk of disease: one variant tagged by rs6502867 and the other located further upstream, in the proximal coding region or in the transcriptional promoter.

In one example, the markers rs878329, rs7223628, rs8182352, and rs4790796 are in near perfect linkage disequilibrium with rs4790797 (data not shown), indicating that these five SNPs, which span only 2107 bases, all represent the same signal. The colinearity among the five markers precludes logistic-regression analyses that include any two of them. Inclusion of rs4790797 significantly improved the fit of models that included any 1 of the 14 remaining markers, except for rs12150220 and rs2670660, whereas none of the 14 remaining markers, except for rs6502867, improved the fit of the model that included rs4790797. These results suggest that an association of 11 of the 14 markers (rs961826, rs11078575, rs1877658, rs925597, rs925598, rs3926687, the 12-bp deletion, rs2733359, rs35658367, rs2716914, and rs8182354) with disease may be secondary to linkage disequilibrium with the cluster of 5 SNPs represented by rs4790797. Overall, in one particular example, a marker most significantly associated with disease was rs4790797. The results for the vitiligo phenotype were similar to those for the expanded autoimmune and autoinflammatory disease phenotype, except that the association of rs12150220 with vitiligo also appeared to be secondary to linkage disequilibrium with rs4790797.

Thus, at least two independent signals were detected associated with autoimmune and autoinflammatory diseases: one located within the NALP1 structural gene, tagged by SNP rs6502867, and at least one other, located within a 64.7-kb linkage disequilibrium block tagged by the nonsynonymous coding SNP rs12150220 (Leu155→His) and six promoter-region SNPs (rs2670660, rs878329, rs7223628, rs8182352, rs4790796, and rs4790797). The significance (P=0.0001) of a model that includes two SNPs, rs6502867 and rs4790797, each of which represents one of the two independent association signals, was greater than that of either individual SNP (P<0.001 for each), and the haplotype carrying both high-risk alleles conferred the highest risk (odds ratio, 2.77; P<0.001) (Table 11).

In one exemplary method, the haplotype-specific effects of rs6502867 and rs4790797 (representing the cluster of five SNPs with perfect linkage disequilibrium) were assessed by comparing logistic-regression models that included the additive effects of both loci, with and without accounting for linkage phase. In this example, no significant haplotype-specific effects were identified, which implies that there is little or no difference whether the two variants in the NALP1 region are cis or trans to one another. In addition, logistic-regression models were used to evaluate the mode of inheritance of risks individually associated with rs6502867 and rs4790797. These analyses favored an additive model with no dominant or recessive effects.

### Analysis of NALP1 Nonsynonymous Coding Region Variants

In one exemplary method, a total of 15 SNPs in the NALP1 region predicted nonsynonymous amino acid substitutions in the NALP1 protein (Table 9). These 15 SNPs were included in the second set of 78 SNPs tested for an association with disease in the 114 families (FIG. 1C), and only rs12150220 (Leu155→His) showed evidence of an association, both with vitiligo alone and with autoimmune and autoinflammatory diseases (P=0.002 and P=0.001, respectively, by the pedigree disequilibrium test). Leu155→His is a nonconservative substitution located between the N-terminal pyrin and NACHT domains of the human NALP1 polypeptide. This region contains no known peptide motifs, but its amino acid sequence, including residue Leu155, has been highly conserved through primate evolution, from bush baby to human—suggesting that the region is critical to protein function. Indeed, the predicted secondary structure of the region has been even more highly conserved than its sequence.

### NALP1 Promoter-Region Variants

In one exemplary study, 205 variants in the extended promoter region were examined, all of which were assessed for predicted effects on transcription-factor binding motifs. Five of the six tightly linked SNPs in the promoter region that were associated with disease were found to affect high-probability mammalian transcription-factor binding sites (Table 3). Furthermore, rs2670660 occurs within a segment that is remarkably conserved in the human, chimpanzee, macaque, bush baby, cow, mouse, and rat, suggesting that this variant is functionally significant. It alters predicted binding motifs for the transcription factors HMGA1 [HMG-I(Y)] and MYB. MYB regulates transcription during the differentiation, proliferation, and apoptosis of erythroid, myeloid, and lymphoid cell lineages. Whether any of these sequence variants affects NALP1 transcription in humans requires further investigation.

In one exemplary FIGURE, FIG. 1. association of Autoimmune Disease with the Chromosome 17 Linkage Region and NALP1 is represented. Panel A represents an exemplary nonparametric multipoint lod scores for markers across chromosome 17 for 51 multiplex families with vitiligo-associated multiple autoimmune and autoinflammatory disease. Panel B represents an exemplary distribution of 177 single-nucleotide polymorphisms (SNPs) genotyped across the 17p linkage region, from 0.04 to 6.24 Mb, in the same 51 families. The 23 SNPs that were each significantly associated with disease in these 51 families (circles) were then genotyped in a replicate series of 63 similar families (circles indicate SNPs that were significantly associated with disease in these families) (see Table 7). Panel C represents an exemplary negative log 10 of the P values from the pedigree disequilibrium test for the 85 variants (83 SNPs and 2 insertion-deletion polymorphisms) genotyped across the NALP1 region of chromosome 17p in all 114 families. X's indicate the vitiligo phenotype and O's indicate the extended autoimmune and autoinflammatory disease phenotype. The 18 exons of the NALP1 structural gene are indicated by the vertical bars (transcriptional orientation shown from right to left; arrow). Panel D shows pairwise r2 values for linkage disequilibrium (with darker boxes indicating stronger disequilibrium) among the 19 of the 85 NALP1 region markers shown in Panel C that were most consistently associated with disease and thus were used in conditional logistic-regression analyses, graphed against the physical positions of the markers. Stars indicate markers for which potential independent effects could not be excluded through regression analysis.

### Examples 2 and 3

In another exemplary study, association of high-risk NALP1 region SNPs with vitiligo was independently confirmed in a series of Romanian patients with generalized vitiligo (Tables 4 and 5). Four of the NALP1 region SNPs were selected that were highly associated with risk of vitiligo and other autoimmune diseases (rs6502867, rs12150220, rs2670660, rs8182352) in the multiple autoimmune disease families. These four SNPs were genotyped in 66 Romanian patients with generalized vitiligo (many with other vitiligo-associated autoimmune and autoinflammatory diseases) and in 93 population-matched controls who did not have vitiligo or any other autoimmune or autoinflammatory diseases. Allele frequencies for each SNP were then compared in cases versus controls, as were genotype frequencies and overall genotype distributions. As represented in exemplary Table 4, all four of these SNPs showed association with generalized vitiligo in the Romanian patients by one or more of these measures, and for the same high-risk SNP alleles found previously. Combinatorial risk was then assessed considering rs6502867 and rs12150220 as tags for the two independent NALP1 risk variants detected previously. As shown in exemplary Table 5, in the Romanian patients risk was greatest (odds ratio 3.65; 94% confidence interval 1.53-8.29) when patients had high-risk alleles of both of these two SNPs. Taken together, these results independently confirm that NALP 1-region variants specifically confer risk of vitiligo, that there are at least two independent variants in the gene, and that highest risk is associated with presence of both variants.

In another exemplary study, association of high-risk NALP1 region SNPs with latent autoimmune diabetes in adults (LADA; also sometimes called type 1.5 diabetes mellitus) were tested. LADA is an important form of adult diabetes mellitus, in which autoimmune diabetes similar to that in childhood has onset during adulthood. LADA is thought to account for about 10% of all adult diabetes mellitus, and thus is far more frequent than typical childhood type 1A diabetes mellitus. LADA patients typically exhibit slowly-progressive autoimmune diabetes, and most patients manifest GAD65 autoantibodies; most patients ultimately progress to requiring insulin for control of their diabetes. Previously, LADA was shown to be epidemiologically associated with vitiligo, and that LADA is part of the extended autoimmune/autoinflammatory disease phenotype that is genetically associated with NALP1 region SNPs. To specifically test whether NALP1 SNPs are associated with LADA, four NALP1 region SNPs were selected that are highly associated with risk of vitiligo and other autoimmune diseases (rs6502867, rs12150220, rs2670660, rs8182352), as well as four NALP1 region SNPs not associated with disease risk (rs8074853, rs16954840, rs11078587, rs1008588), which are not highly associated with risk of vitiligo. These SNPs were genotyped in 15 Dutch GAD65-positive LADA patients and 121 population-matched controls with typical type 2 diabetes mellitus. Allele frequencies for each SNP were then compared in cases versus controls, and the entire dataset was evaluated by conditional logistic regression analysis. As shown in Table 6, SNP rs6502867, which had previously been found to be highly associated with risk of vitiligo and other autoimmune diseases, was likewise highly associated with LADA in the Dutch patients, with an odds ratio of 4.04 (95% confidence interval 0.92-17.66). SNP rs2670660 also came close to significance. None of the other SNPs showed association. Taken together, these results indicate that intragenic NALP1 SNP rs6502867, and perhaps also promoter region SNP rs2670660, is associated with risk of LADA.

### Example 4

### Genetic Variations in NALP1 Gene Associated with Autoimmune/Autoinflammatory Disease

High density SNP genotyping was used to perform a detailed genetic linkage analysis and to identify a gene, haplotypes and specific genetic variations in apparent association with vitiligo-associated multiple autoimmune/autoinflammatory disease. Single-nucleotide polymorphism (SNP) genotyping and pedigree-based association analysis in 114 multiplex families with vitiligo-associated multiple autoimmune/autoinflammatory disease were used to identify the NALP1 gene as putatively causal for the disease complex. DNA re-sequencing was performed to identify disease-associated sequence variants and haplotypes.

Fine-scaled linkage and association mapping in an extended multiple autoimmune disease family cohort confirmed linkage and association of both vitiligo alone and vitiligo-associated multiple autoimmune/autoinflammatory disease to a high-risk SNP haplotype spanning the proximal portion of the NALP1 gene through at least 68.3 kbp of the proximal NALP1 promoter. We identified specific variants in the NALP1 promoter that may mediate disease susceptibility

### Methods

DNA Sequencing. In one example, 76 kbp of the NALP1 genomic region was sequenced in 4 European or American Caucasian individuals heterozygous for the high-risk NALP1 haplotype 1 who, though themselves unaffected by autoimmune disease, had transmitted the high-risk haplotype to at least one affected offspring; two were from cohort 1 and two were from cohort 2. The regions sequenced included contiguous 69.1 kbp spanning the extended NALP1 promoter and exons 1-3, and four individual segments containing exons 4-7 and 90-160 bp of adjacent intronic sequences. Approximately 67% of the region was sequenced on both DNA strands. Sequence variants for which all four haplotype 1 carriers were heterozygous were then re-sequenced in one individual with vitiligo-associated multiple autoimmune/autoinflammatory disease, the parent of one of the four heterozygotes, who was homozygous for the NALP1 disease-associated proximal haplotype. The predicted effects of both alleles of all promoter variants carried on the NALP1 haplotype 1 on potential transcription factor binding motifs were assessed by use of an online Transcription Element Search System (TESS) (Schug J, Overton C. TESS: Transcription Element Search Software on the WWW. Technical Report CBIL-TR-1997-1001-v0.0, Univ Pennsylvania 1997. URL: cbil.upenn.edu/TESS). Nucleotide sequence positions were taken from the NCBI Build 35.1 human chromosome 17 sequence.

Allele Frequency Determination. The frequency of a 12-nt deletion in the NALP1 promoter region (nt 5,457,169-5,457,180) was determined by PCR by amplifying a 105-bp product spanning the deletion, using as primers 5′-TGTGTTCTCAAGTGCCTTATG C-3′ (SEQ ID NO:2) and 5′-CATCTTTGCTCGTGTGTGTG-3′ (SEQ ID NO:3), and analyzing the products by electrophoresis in 5% polyacrylamide gels. Chi-square tests were performed to assess Hardy-Weinberg equilibrium of the deletion in 95 unrelated normal controls of northern European Caucasian ethnic origin.

### Statistical Analyses

Preliminary Analyses. In one exemplary method, inheritance of each marker was assessed in all families, using PedCheck software (O'Connell and Weeks, 1998, Am J Hum Genet. 63:259-66), to test for inconsistencies due to non-paternity, new mutations, or other errors.

Genetic Linkage Analysis. For genetic linkage analysis of chromosome 17, microsatellite genotype data for the 51 families that previously showed linkage to 17p (Spritz et al., 2004) were re-analyzed using Allegro 1.2c software (Karason et al., 2003, Am J Hum Genet. 72:125-31) using an exponential model under the Sₚₐᵢᵣₛ scoring function after applying the error-checking routine implemented in Merlin 1.0.0 (Abecasis et al., 2002, Nature Genet. 30:97-101).

Tests of Hardy-Weinberg Equilibrium. In another method, Hardy-Weinberg equilibrium in founders and persons not in the line of descent, such as spouses, in all 114 families were tested. To investigate the possibility of transmission distortion unrelated to disease (Spielman et al., 1993, Am J Hum Genet. 52:506-16), we used the TDTPHASE command implemented in the UNPHASED program (Dudbridge, 2003 Genet Epidemiol 25:115-21), version 2.403, to examine the transmission of alleles of each NALP1 region SNP that yielded significant P values (P<0.05) to unaffected siblings in nuclear families derived from our extended families.

Analyses of Single Nucleotide Polymorphisms and Haplotype Association. All families studied are multiplex families with both vitiligo and vitiligo-associated autoimmune/autoinflammatory disease. To determine whether NALP1 region SNPs are associated with vitiligo itself, or with vitiligo-associated multiple autoimmune/autoimmune disease as a whole, we coded “affected” in two ways: either just family members with vitiligo were coded as “affected” or, alternatively, anyone with any of the eight vitiligo-associated autoimmune/autoinflammatory diseases (vitiligo, autoimmune thyroid disease, rheumatoid arthritis, adult-onset autoimmune diabetes mellitus, psoriasis, pernicious anemia, systemic lupus erythematosus, and Addison's disease) were coded as “affected”. Individual nominal P values for association with vitiligo or with all vitiligo-associated autoimmune/autoinflammatory diseases were calculated for each SNP with the use of the Pedigree Disequilibrium Test (PDT) (Martin et al., 2000, Am J Hum Genet. 6:146-54) version 5.1 and the Family-Based Association Test (FBAT) (Horvath et al., 2004, Genet Epidemiol 26:61-9) version 1.5.5. To calculate the odds ratios (OR) for each SNP we used TDTPHASE of the UNPHASED program (Dudbridge, 2003) to count the transmitted and untransmitted alleles.

Haplotype-based transmission-disequlibrium statistics were also calculated using FBAT 1.5.5. Haplotypes were determined by conditioning on the sufficient statistic, which was based on observed parental and offspring genotypes. To calculate odds ratios for the high-risk haplotype versus all other haplotypes we used TDTPHASE of the UNPHASED program (Dudbridge, 2003) to count the transmitted and untransmitted haplotypes.

Testing of Linkage Disequilibrium Between SNPs. Calculation of linkage disequilibrium between NALP1 region SNPs and derivation of haplotype block structures was carried out using Haploview (Barrett et al., 2005, Bioinformatics 21:263-65) version 3.2. Calculation of association of each SNP with vitiligo or with all vitiligo-associated autoimmune/autoinflammatory diseases (considering as “affected” all individuals with any of these disorders) was carried out using the Pedigree Disequilibrium Test (PDT) (Martin et al., 2000, Am J Hum Genet. 6:146-54) version 5.1 and the Family-Based Association Test (FBAT) (Horvath et al., 2004, Genet Epidemiol 26:61-9) version 1.5.5. Permutation-based PDT P values were calculated using PTDPHASE of the UNPHASED program (Dudbridge, 2003, Genet Epidemiol 25:115-21). Haplotype-based transmission-disequilibrium statistics were calculated using FBAT 1.5.5. Odds ratios (OR) for individual SNPs and SNP haplotypes were calculated using TDTPHASE of the UNPHASED program. To calculate genotype relative risk for vitiligo and associated autoimmune/autoinflammatory diseases due to inheritance of zero, one, or two copies of the high-risk haplotype, we used as the patient group probands from 104 of the 114 families in which we could determine haplotype phase, as one control group founders and marrying-in relatives from the same 104 families, and as another control group so-called “affected family based controls” (AFBAC) (Thompson G, 1995, Am J Hum Genet. 57:487-98).

### Exemplary Results

As represented in FIG. 1B, family-based joint linkage and association analysis identified one replicable association signal in the chromosome 17p linkage peak, defined a cluster of three adjacent SNPs—rs3926687, rs2733359, and rs878329—with significant individual nominal P values (P≦0.05) for vitiligo alone, and for the entire group of autoimmune/autoinflammatory diseases. The SNPs within this cluster were in Hardy-Weinberg equilibrium in the founders and marrying-in spouses, and the three SNP clusters showed no linkage disequilibrium with each other. This SNP cluster encompasses a 117 kbp segment that includes the extended promoter region and most of the coding region of a compelling biological candidate gene, NALP1, which encodes a key regulator of the innate immune system. In fact, this genomic region is remarkably gene-sparse; SNPs located downstream of NALP1 were not significantly associated, and the closest upstream gene, KIAA0523, is located more than 486 kbp centromeric of NALP1. Analysis of haplotypes derived from these three adjacent SNPs showed that, in both the 51-family series and the independent 63-family series, both series of families with VAAAD, and in the combined 114-family series, the most significant disease association, both for vitiligo alone and for the entire group of vitiligo-associated autoimmune/autoinflammatory diseases, was to a three-SNP haplotype defined by rs3926687-rs2733359-rs878329, here termed “haplotype 1” [51-family series: vitiligo, P=0.031, autoimmune/autoinflammatory disease, P=0.033; 63-family series: vitiligo, P=0.034, autoimmune/autoinflammatory disease, P=0.016; combined 114-family series: vitiligo, P=0.0026, autoimmune/autoinflammatory disease, P=0.0014]. Haplotype 1 encompassed about 61 kbp of the NALP1 extended promoter region, indicating that variation within NALP1 or its extended promoter is responsible for the 17p linkage signal corresponding to SLEV1.

In one exemplary method to localize the autoimmunity/autoinflammatory disease susceptibility mutation in the NALP1 region, an association study was expanded by determining the genotypes in all 114 families for 85 additional SNPs distributed across the NALP1 gene and 246 kbp of its adjacent 5′ promoter region (Table 4), As shown in FIG. 1C, many of these variants were significantly associated with vitiligo, with associated autoimmune/autoinflammatory diseases, or both, in a pattern broadly distributed across the proximal portion of the NALP1 structural gene and its extended promoter. Outside of this region disease association declines rapidly (Table 7), indicating that the causal variants are within NALP1 structural gene and/or its extended promoter region.

Of the total 85 SNPs genotyped in the NALP1 region, none with genotype P values <0.05 showed significant deviation from Hardy-Weinberg equilibrium (data not shown). Transmission disequilibrium testing of all SNPs with significant disease association (P<0.05), using control samples comprised of unaffected siblings of nuclear families from the 114 extended families, showed no significant transmission distortion.

As determined by DNA sequence analysis of sequence analysis of 82.9 kb of NALP1 and its extended promoter region in each of 11 persons with vitiligo who were homozygous for haplotype 1 and in 4 unaffected heterozygotes who had transmitted haplotype 1 to at least one affected offspring, we discovered that the disease-associated high-risk haplotype 1 carries several novel deletions (Table 8), the largest of which is a 12 bp deletion (nt 5,457,169-5,457,180 delTATGACTATGTG (SEQ ID NO:1)) located adjacent to a highly conserved 250 bp sequence located approximately 29.2 kbp upstream of the NALP1 transcriptional start site. This deletion abolishes several potential transcriptional motifs, including one of a remarkable (P=9.94e−231) cluster of six potential binding motifs for locus control region factor 1 (LCR-F1, TCF-11, Nrf1), suggesting that this region might constitute a locus control element for regulating NALP1 expression.

These findings establish NALP1 as a broad-spectrum autoimmune/autoinflammatory disease susceptibility gene, along with HLA, CTLA4, and PTPN22 (Brand et al., 2005), which mediate aspects of the adaptive immune response. The identification of NALP1 as a broad-spectrum autoimmunity/autoinflammatory disease susceptibility gene implicates the innate immune system in pathogenesis of these diseases, providing important new insights into potential gene-based and biomarker-based diagnosis of disease, pre-symptomatic disease susceptibility, therapeutics, and disease prevention. It will high-risk variants in the different broad-spectrum susceptibility genes interact with each other, interact with disease-specific susceptibility genes, and interact with environmental triggers to promote induction and establishment of autoimmune diseases.

The results provided herein indicate that NALP1 plays an important role in autoimmune/autoinflammatory disease. NALP1 may in fact regulate the activity of the entire caspase pathway. Assays to detect genetic variations in NALP1 associated with such diseases are of use to detect, diagnose or predict the risk of developing vitiligo associated autoimmune/autoinflammatory disease. Further, NALP1 or its downstream pathway partners may provide therapeutic targets for specific treatment and/or prevention of autoimmune/autoinflammatory disease.

TABLE 1
Autoimmune and autoinflammatory disease
phenotypes in 114 multiplex families*

| Disease phenotype | Number of cases |
|---|---|
| Vitiligo only | 219 |
| Vitiligo and autoimmune thyroid disease | 70 |
| Vitiligo, autoimmune thyroid disease, and other, | 20 |
| non-thyroid autoimmune disease | |
| Vitiligo and other autoimmune disease | 60 |
| Autoimmune thyroid disease only | 86 |
| Autoimmune thyroid disease + other autoimmune | 23 |
| disease | |
| Other autoimmune disease only | 89 |

*A total of 567 study family members reported at least one autoimmune or autoinflammatory disease; 175 reported more than one disease.

TABLE 2
Association of 19 NALP1 variants with vitiligo and autoimmune and inflammatory
disease in 114 multiplex families¹

| | P Values | | | | |
|---|---|---|---|---|---|
| | Pedigree | Family-Based | Conditional | | |
| | Disequilibrium Test | Association Test | Logistic- | | 95% |
| Variant/allele | (PDT) | (FBAT) | Regression | Odds Ratio | Confidence Interval |
| Autoimmune and | | | | | |
| autoinflammatory | | | | | |
| Disease | | | | | |
| rs6502867/A | 0.0005 | 0.0022 | 0.0006 | 1.93 | 1.32-2.88 |
| rs961826/A | 0.0012 | 0.0012 | 0.0075 | 1.60 | 1.14-2.24 |
| rs12150220/A | 0.0010 | 0.0012 | 0.0033 | 1.66 | 1.19-2.31 |
| rs11078575/C | 0.0027 | 0.0021 | 0.0157 | 1.50 | 1.08-2.08 |
| rs1877658/T | 0.0025 | 0.0050 | 0.0446 | 1.40 | 1.01-1.93 |
| rs925597/A | 0.0005 | 0.0005 | 0.0054 | 1.62 | 1.16-2.27 |
| rs925598/A | 0.0014 | 0.0016 | 0.0040 | 1.63 | 1.17-2.26 |
| rs3926687/T | 0.0020 | 0.0026 | 0.0059 | 1.59 | 1.15-2.21 |
| 12 bp del | 0.0063 | 0.0139 | 0.0129 | 1.51 | 1.10-2.09 |
| rs2670660/C | 0.0007 | 0.0004 | 0.0031 | 1.68 | 1.20-2.35 |
| rs2733359/G | 0.0023 | 0.0013 | 0.0053 | 1.64 | 1.17-2.30 |
| rs35658367/ATGA | 0.0013 | 0.0010 | 0.0044 | 1.64 | 1.17-2.31 |
| rs2716914/C | 0.0031 | 0.0036 | 0.0173 | 1.49 | 1.08-2.07 |
| rs878329/G | 0.0045 | 0.0012 | 0.0029 | 1.63 | 1.17-2.26 |
| rs7223628/G | 0.0024 | 0.0007 | 0.0015 | 1.68 | 1.21-2.35 |
| rs8182352/G | 0.0013 | 0.0004 | 0.0004 | 1.81 | 1.28-2.56 |
| rs4790796/A | 0.0026 | 0.0007 | 0.0010 | 1.73 | 1.24-2.42 |
| rs4790797/T | 0.0023 | 0.0005 | 0.0003 | 1.82 | 1.29-2.56 |
| rs8182354/A | 0.0040 | 0.0010 | 0.0015 | 1.69 | 1.22-2.36 |
| Vitiligo | | | | | |
| rs6502867/A | 0.0047 | 0.0038 | 0.0006 | 2.08 | 1.37-3.15 |
| rs961826/A | 0.0024 | 0.0028 | 0.0067 | 1.67 | 1.15-2.41 |
| rs12150220/A | 0.0022 | 0.0019 | 0.0041 | 1.69 | 1.18-2.41 |
| rs11078575/C | 0.0050 | 0.0048 | 0.0153 | 1.55 | 1.09-2.19 |
| rs1877658/T | 0.0040 | 0.0071 | 0.0266 | 1.49 | 1.04-2.1 |
| rs925597/A | 0.0019 | 0.0025 | 0.0048 | 1.69 | 1.18-2.44 |
| rs925598/A | 0.0033 | 0.0027 | 0.0051 | 1.66 | 1.16-2.36 |
| rs3926687/T | 0.0043 | 0.0041 | 0.0077 | 1.61 | 1.14-2.29 |
| 12 bp del | 0.0011 | 0.0089 | 0.0070 | 1.66 | 1.16-2.36 |
| rs2670660/C | 0.0004 | 0.0011 | 0.0021 | 1.80 | 1.25-2.61 |
| rs2733359/G | 0.0013 | 0.0042 | 0.0037 | 1.75 | 1.21-2.53 |
| rs35658367/ATGA | 0.0004 | 0.0011 | 0.0021 | 1.82 | 1.24-2.67 |
| rs2716914/C | 0.0015 | 0.0075 | 0.0072 | 1.64 | 1.15-2.35 |
| rs878329/G | 0.0017 | 0.0030 | 0.0021 | 1.75 | 1.22-2.51 |
| rs7223628/G | 0.0007 | 0.0015 | 0.0010 | 1.82 | 1.26-2.63 |
| rs8182352/G | 0.0004 | 0.0008 | 0.0002 | 2.01 | 1.36-2.95 |
| rs4790796/A | 0.0011 | 0.0021 | 0.0010 | 1.83 | 1.26-2.63 |
| rs4790797/T | 0.0007 | 0.0011 | 0.0002 | 2.01 | 1.39-2.91 |
| rs8182354/A | 0.0014 | 0.0022 | 0.0011 | 1.83 | 1.27-2.64 |

*The variants listed are those for which the association with disease was replicated by the in PDT and the FBAT in both series of families. Odds ratios (OR) were calculated from the coefficients of the regression equation. The 12 bp deletion (12 bp del) includes nucleotides 5457169 to 5457180 (TATGACTATGTG (SEQ ID NO: 1)).

TABLE 3
Predicted transcription-factor binding motifs affected by associated
NALP1 promoter-region SNPs associated with disease*

| SNP/allele | Nucleotide position | Transcription Factor | Lₐ | L_{q} |
|---|---|---|---|---|
| rs2670660 | 5,459,730 | | | |
| T | | HMGA1 [HMG-I(Y)] | 22.00 | 0.917 |
| C(high-risk) | | MYB | 12.00 | 1.000 |
| rs878329 | 5,493,974 | | | |
| C | | PEBP2 | 18.00 | 0.900 |
| G (high-risk) | | — | | |
| rs7223628 | 5,495,192 | | | |
| A | | AP-1 | 18.00 | 0.900 |
| | | NFAT-1 | 14.00 | 1.000 |
| G (high risk) | | TCF2 (HNF1B) | 14.00 | 1.000 |
| | | PU.1 | 14.00 | 1.000 |
| rs8182352 | 5,495,711 | | | |
| A | | — | | |
| G (high-risk) | | PR | 12.00 | 1.000 |
| rs4790797 | 5,496,043 | | | |
| C | | FOXF1 | 24.00 | 0.923 |
| T (high risk) | | — | | |

*The five SNPs are among the six NALP1 promoter-region SNPs whose potential independent effects could not be ruled out by conditional logistic-regression analyses. Lₐ denotes the log-likelihood score. L_{q}, a measure of the goodness-of-fit of the DNA sequence to the consensus binding motif, was calculated by dividing Lₐ by the maximum Lₐ possible for the site model; the best possible Lₐ was 1.000. The NALP1 exon 1 (including the site of start of translation) begins at nucleotide 5,428,550.

TABLE 4
Allele and genotype distributions of NALP1 SNPs in the Romanian
Vitiligo patients and the healthy, unrelated controls

| | | | | |
|---|---|---|---|---|
| Cases | Controls | OR (95% CI) | P-value | P-value |

[G/A] rs6502867

| | | | | | |
|---|---|---|---|---|---|
| GG | 3 (0.05) | 8 (0.09) | 1.0 (referent) | | |
| GA | 18 (0.27) | 39 (0.42) | 1.23 (0.29-5.19) | 0.777 | |
| AA | 45 (0.68) | 46 (0.49) | 2.61 (0.65-10.46) | 0.176 | 0.058 |

| | | | |
|---|---|---|---|
| A % | 81.8 | 70.4 | 0.021 |

[T/A] rs12150220

| | | | | | |
|---|---|---|---|---|---|
| TT | 9 (0.14) | 33 (0.35) | 1.0 (referent) | | |
| TA | 38 (0.57) | 33 (0.35) | 4.22 (1.77-10.10) | 0.001 | |
| AA | 19 (0.29) | 27 (0.29) | 2.58 (1.01-6.62) | 0.049 | 0.003 |

| | | | |
|---|---|---|---|
| A % | 57.6 | 46.8 | 0.070 |

[T/C] rs2670660

| | | | | | |
|---|---|---|---|---|---|
| TT | 9 (0.14) | 29 (0.31) | 1.0 (referent) | | |
| TC | 34 (0.51) | 37 (0.40) | 2.96 (1.23-7.15) | 0.016 | |
| CC | 23 (0.35) | 27 (0.29) | 2.74 (1.08-6.97) | 0.034 | 0.032 |

| | | | |
|---|---|---|---|
| C % | 60.6 | 48.9 | 0.048 |

[A/G] rs8182352

| | | | | | |
|---|---|---|---|---|---|
| AA | 8 (0.12) | 26 (0.28) | 1.0 (referent) | | |
| AG | 38 (0.58) | 39 (0.42) | 3.17 (1.28-7.86) | 0.013 | |
| GG | 20 (0.30) | 28 (0.30) | 2.32 (0.87-6.18) | 0.092 | 0.034 |

| | | | |
|---|---|---|---|
| G % | 59.1 | 51.1 | 0.0161 |

P values were obtained from Wald test of logistic regression model; odds ratios (ORs) were calculated from the coefficients of the regression equation

TABLE 5
Risk of Vitiligo associated with possession of two NALP1 variants

| | Vitilgo cases | Controls, | | |
|---|---|---|---|---|
| Variant status | N(%) (n = 66) | N(%) (n = 93) | OR (95% CI | P |
| Wild type or possessing high risk allele(s | 9(13.6) | 34(36.6) | 1.0(referent) | |
| from either rs6502867 or rs12150220 | | | | |
| Possessing high risk allele(s) from both | 57(86.4 | 59(63.4) | 3.65(1.53-8.29) | 0.001 |
| rs6502867 and rs12150220 | | | | |

OR, odds ratio; CI, confidence interva

TABLE 6
Association of NALP1 region SNPs with in Dutch LADA patients

| | | | Fisher's P | | | | | |
|---|---|---|---|---|---|---|---|---|
| | | | value on | Chi-square | | logistic | | |
| | Associated | Case, Control | chromsomal | OR for high | | df(1) | OR from co- | |
| SNP | Allele | Ratios | level | risk allele | 95% CI | P value | efficient | 95% CI |
| rs6502867 | 1 | 28:2, 188:54 | 0.030 | 4.02 | 0.89-25.26 | 0.026 | 4.04 | 0.92-17.66 |
| rs8074853 | 2 | 16:14, 95:147 | 0.100 | 1.77 | | 0.131 | | |
| rs16954840 | 2 | 6:24, 21:219 | 0.060 | 2.63 | 0.85-7.72 | 0.070 | 2.78 | 0.97-7.98 |
| rs12150220 | 1 | 17:13, 107:133 | 0.145 | 1.63 | | 0.228 | | |
| rs2670660 | 2 | 18:10, 109:129 | 0.054 | 2.13 | 0.89-5.2 | 0.068 | 2.11 | 0.92-4.84 |
| rs8182352 | 2 | 15:11, 116:126 | 0.230 | 1.48 | | 0.343 | | |
| rs11078587 | 2 | 19:9, 144:98 | 0.260 | 1.44 | | 0.354 | | |
| rs1008588 | 2 | 18:10, 125:113 | 0.163 | 1.63 | | 0.230 | | |

SNPs in bold are selected as highly associated with risk of vitiligo and other autoimmune diseases

TABLE 7
²P Values for Allelic Association of 17p SNPs with Vitiligo and Extended
Autoimmune/Autoinflammatory Disease Phenotype

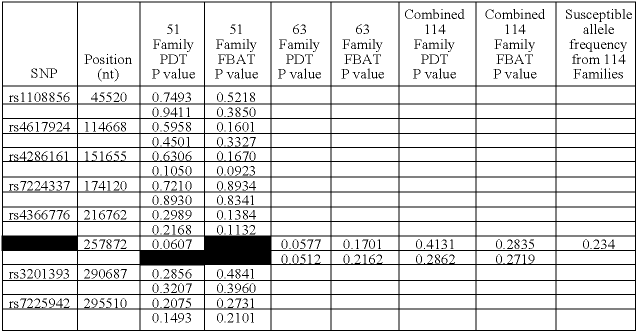

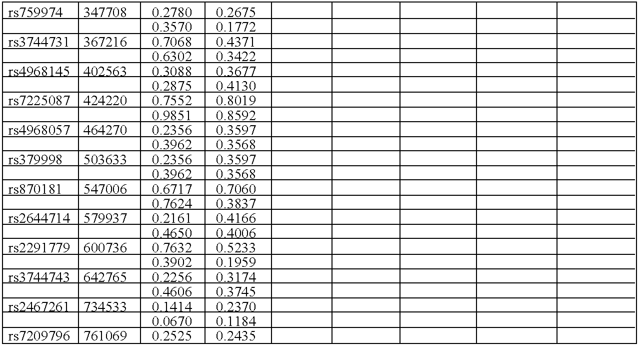

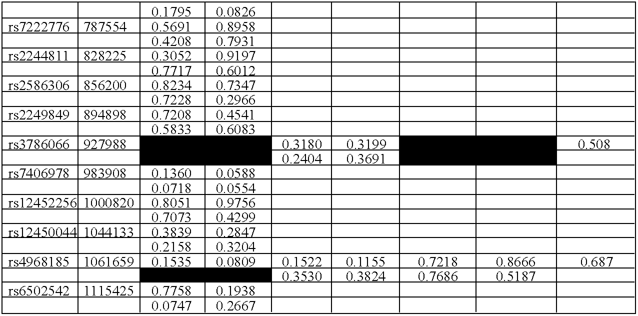

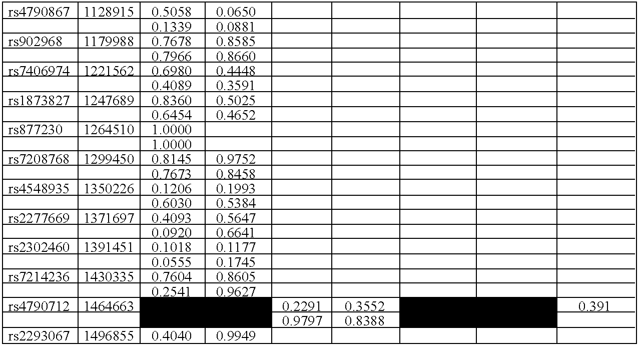

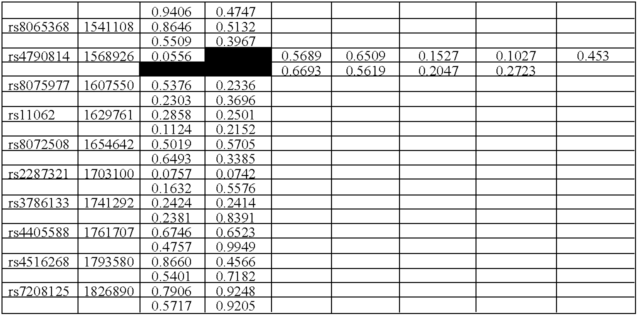

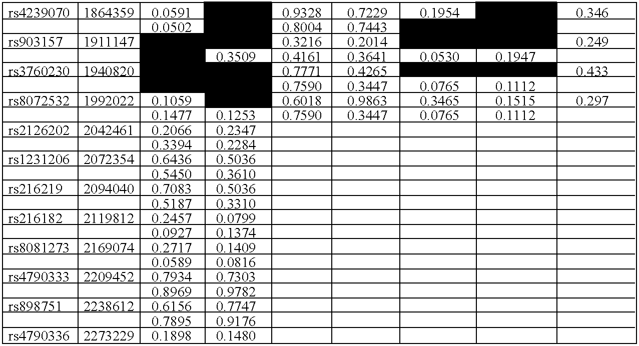

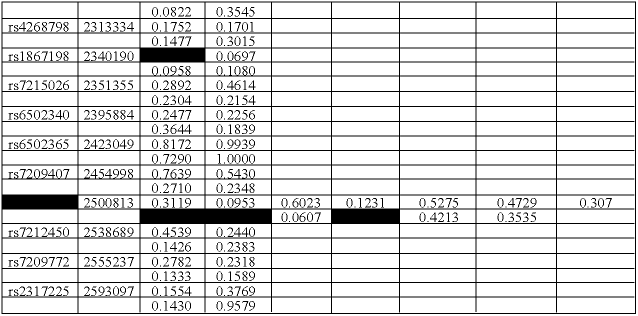

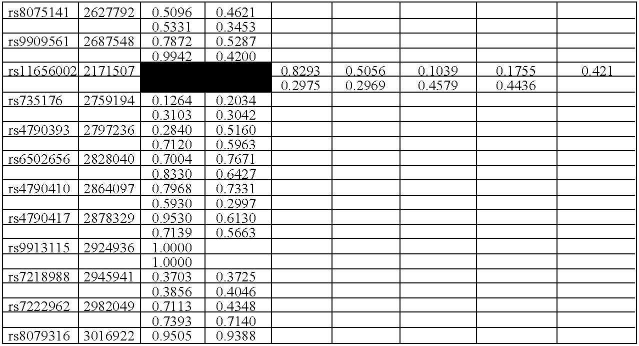

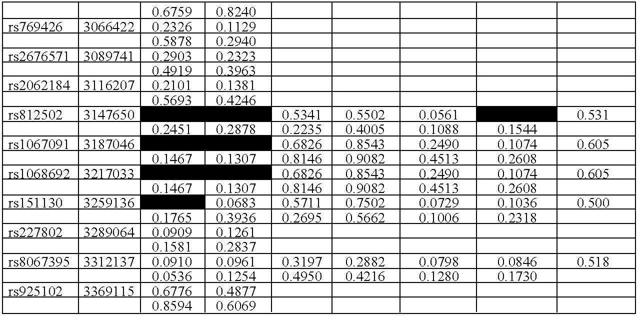

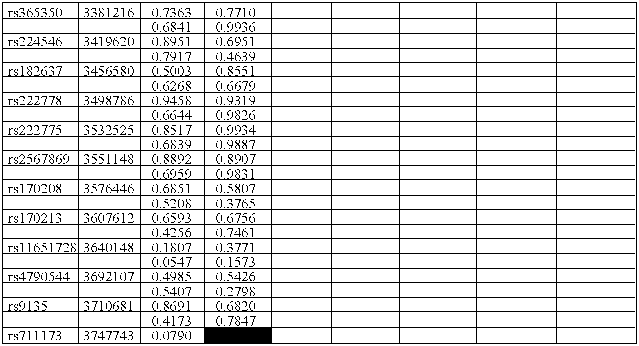

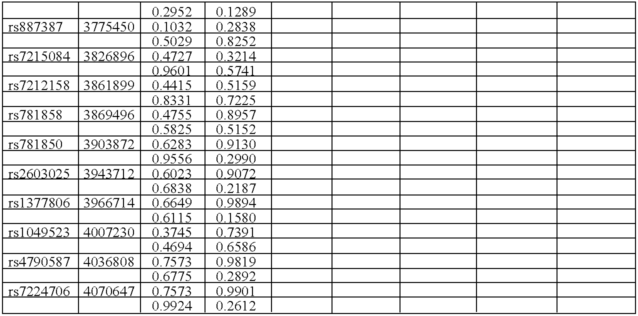

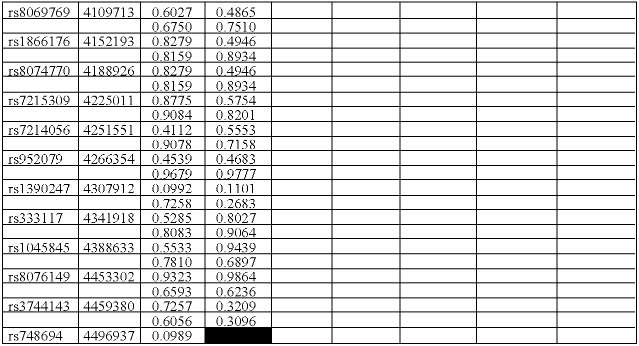

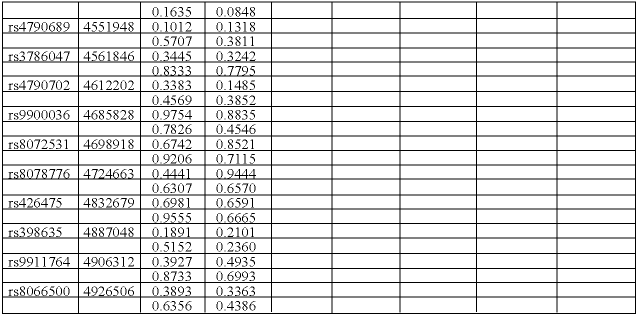

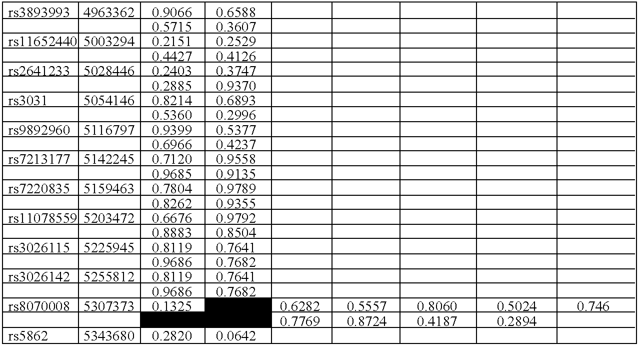

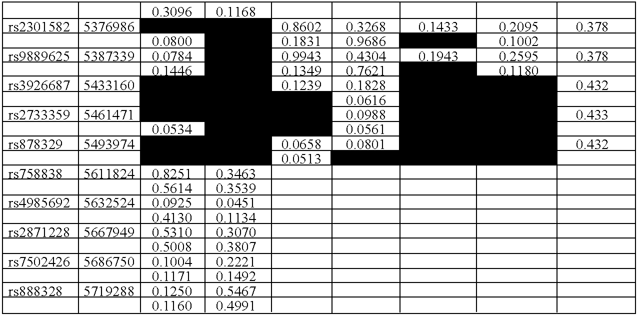

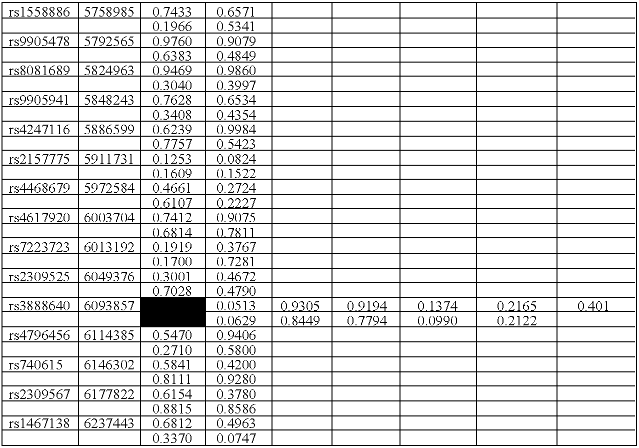

Regular type: vitiligo phenotype;
Italicized: extended autoimmune/autoinflammatory disease phenotype.
Shaded cells: nominal P value <0.05;
Diagonal stripe cells: markers showing association with opposite alleles in cohorts 1 and 2.

³TABLE 8
NALP1 Region Variants Identified by DNA Sequence Analysis

| ENSEMBL | | | | | | Observed |
|---|---|---|---|---|---|---|
| NT # | Haplotype 1 | Haplotype 2 | Haplotype 3 | Unknown | rs# | Allele Freq* |
| 5345721 A | A and G | A | A | | novel | q = 1/28 |
| 5346065 C | C and G | C | C | | rs3809845 | |
| 5359384 G | G | G | G | A (1 or 2?) | novel | q = 1/24 |
| 5359384 C | C and T | C | C | | rs2137722 | |
| 5360002 G | G and A | G and A | G | | rs9900365 | |
| 5360609 G | G and A | G | G | | novel | q = 5/30 |
| 5361052 G | G and A | G and A | G and A | | rs6502867 | |
| 5361729 A | A and T | T | A and T | | novel | q = 22/30 |
| 5361730 G | G and T | G | T | | novel | q = 22/30 |
| 5364766 A | A and G | A and G | G | | rs12937224 | |
| 5364853 G | G and C | G | G | | rs12946467 | |
| 5365100 T | C and T | T | T | | rs12944976 | |
| 5365311 C | C and T | C | C | | rs11653450 | |
| 5365428 A | A and G | A | A | | rs11650171 | |
| 5365481 A | A and G | A | A | | rs11650176 | |
| 5365496 C | C and A | C | C | | rs11653496 | |
| 5365610 C | C and A | C | C | | rs2301583 | |
| 5365630 G | G and C | G | G | | rs11653832 | |
| 5365715 C | C and T | C | C | | rs11653580 | |
| 5365801 A | A and G | G | G | | rs11651270 | |
| 5374565 T | T and C | T | T | | novel | q = 5/30 |
| 5374690 A | A and G | A | A | | rs35596958 | |
| 5374816 G | G and A | G | G | | rs12940290 | |
| 5376987 G | G and A | G | G and A | | rs2301582 | |
| 5377290 G | G and A | G | G | | rs35291181 | |
| 5377792 A | A and G | A | A | | rs35913493 | |
| 5377841 G | G and A | G | G | | rs35548793 | |
| 5377910 G | G and A | G | G | | rs35568355 | |
| 5378009 C | C and T | C | C | | rs34733791 | |
| 5378173 C | C and T | C | C | | rs11654180 | |
| 5380921 A | A and G | A | A | | rs7215856 | |
| 5380945 A | A and G | A | A | | rs7215868 | |
| 5380972 A | A and G | A | A | | rs11657249 | |
| 5381039 A | A and C | A | A | | rs7216010 | |
| 5381368 A | A and G | A | A | | rs11657416 | |
| 5383514 G | G and A | G | G | | novel; V938M | q = 2/30 |
| 5383648 G | G and A | G | G | | rs7223362 | |
| 5383702 G | G and C | G | G | | rs7223514 | |
| 5383740 T | T and C | T | T | | rs7224061 | |
| 5385967 C | C and T | C | C | | rs11657747 | |
| 5386038 C | C and T | C | C | | rs12942931 | |
| 5397326 C | C and T | C | C | | rs11656080 | |
| 5402314 G | G and A | G | G | | novel | q = 26/30 |
| 5402395 C | C and G | C | C | | novel | q = 4/30 |
| 5402571 C | C and T | C | C | | novel | q = 4/30 |
| 5402619 G | G and T | G | G | | novel | q = 4/30 |
| 5402859 A | A and G | A | A | | rs12950235 | |
| 5402964 G | G and A | G | G | | novel | q = 8/30 |
| 5403378 C | C and T | C | C | | rs2001363 | |
| 5403817 G | G and A | G | G | | novel; E309K | q = 2/24 |
| 5404003 C | C and G | C | C | | rs11651595 | |
| 5426091 T | A | T | A | | rs12150220 | |
| 5426440 C | T | C | T | | rs10852870 | |
| 5426509 T | C | T | C | | rs11078575 | |
| 5427567 G | T | G | T | | rs1877658 | |
| 5428545 G | G and C | G | G | | novel | q = 6/28 |
| 5428744 G | G and C | G | G | | novel | q = 4/30 |
| 5428789 C | G | C | G | | rs925595 | |
| 5428800 G | A and G | G | G | | novel | q = 7/30 |
| 5428823 C | T | T | T | C (2?) | rs925596 | |
| 5428872 G | A | G | A | | rs925597 | |
| 5429076 G | A | G | A | | rs925598 | |
| 5429656 A | A | G | A | | rs2670642 | |
| 5429980 A | G | A | G | | rs1156989 | |
| 5430267 C | T | C | T | | rs1156990 | |
| 5430352 G | G | G | G | A (2?) | novel | q = 1/30 |
| 5430540 A | C | A | C | | rs2716937 | |
| 5430738 A | C | A | C | | rs2670643 | |
| 5431011 G | A | G | A | | rs8073142 | |
| 5431086 T | C | T | C | | rs8073839 | |
| 5431117-5431113 | del | GTTTG | del | | novel | q = 26/28 |
| GTTTG | | | | | | |
| 5431426 A | G | A | G | | rs6502870 | |
| 5433160 C | T | C | T | | rs3926687 | |
| 5434160 G | A | G | A | | rs4790781 | |
| 5435307-5435305 | CAT and del | CAT | CAT | | novel | q = 1/30 |
| CAT | | | | | | |
| 5435783 G | G and A | G | G | | rs11654415 | |
| 5438435 G | T | G | T | | rs1554008 | |
| 5439385 G | G and A | G | G | | novel | q = 1/30 |
| 5439531 G | A | G | G | | rs9914508 | |
| 5439679 A | T | A | T | | rs925593 | |
| 5440054 G | G and A | G | G | | novel | q = 1/30 |
| 5440158 A | A and G | A | A | | novel | q = 1/30 |
| 5443162 A | A and G | A | A | | novel | q = 1/30 |
| 5443385 G | A | A | A | G [(2 and | rs907875 | |
| | | | | 4)?] | | |
| 5443608 T | C | T | T | | rs7215823 | |
| 5443775 G | G | G | G | A [(2 and | rs4790783 | |
| | | | | 4)?] | | |
| 5444603 C | T | C | C | | rs2670636 | |
| 5444682 A | A | A | A | G [(2 and | rs8081956 | |
| | | | | 4)?] | | |
| 5446169 G | A | G | G | | rs2137719 | |
| 5446498 G | A | G | G | | rs11078576 | |
| 5448186 C | T | T | T | C [(2 and | rs2716857 | |
| | | | | 4)?] | | |
| 5449814 C | A | A | A | C [(2 and | rs2716858 | |
| | | | | 4)?] | | |
| 5450982 C | T | T | T | C [(2 and | rs925592 | |
| | | | | 4)?] | | |
| 5450992 C | C | C | C | A (4?) | rs12051585 | |
| 5451053 C | T | C | C | | rs925591 | |
| 5451089 G | G | G | A | | rs12051601 | |
| 5451426 G | G and A | G | G | | novel | q = 2/30 |
| 5452162 G | A | G | A | | rs2670635 | |
| 5453368 A | A and G | A | A | | rs11650206 | |
| 5453979 C | insC | C | insC | | novel | q = 28/30 |
| 5454061 T | C | T | C | | rs2670651 | |
| 5454568 G | G and A | G | G | | novel | q = 6/26 |
| 5455647 A | G | A | G | | rs9908518 | |
| 5455715 C | A | C | A | | rs9900174 | |
| 5455822 T | C | T | C | | rs2716921 | |
| 5456177 A | G | A | G | | rs1018280 | |
| 5456464 G | A | G | A | | rs1018281 | |
| 5456669 G | A | G | G | | rs2733376 | |
| 5457169-5457180 | del 12 bp | — | — | | novel | q = 26/30 |
| 5457241 G | G | G | C | | rs11659063 | |
| 5457684 A | G and A | G | G | | rs35885754 | |
| 5457949 G | A | G | G | | rs2733375 | |
| 5457995 C | T | C | C | | rs2716920 | |
| 5458053 A | G | A | A | | rs2716919 | |
| 5458174 G | A | G | G | | rs2670655 | |
| 5458244 C | G | C | T | | rs2670656 & | T q = 2/30 |
| | | | | | novel T on | |
| | | | | | haplotype 4 | |
| 5458378 G | T | G | G | | rs2716918 | |
| 5458491 T | C | T | T | | rs2670657 | |
| 5458607 C | C and T | C | C | | novel | q = 1/30 |
| 5459078 T | A | T | T | | rs2670658 | |
| 5459197 T | C | T | T | | rs2733374 | |
| 5459424 T | A | T | T | | rs2670659 | |
| 5459614 T | T | T | G | | rs4790784 | |
| 5459730 T | C | T | T | | rs2670660 | |
| 5460531 C | G | C | C | | rs2670661 | |
| 5460550 G | C | G | G | | rs1877659 | |
| 5460953 A | G | A | G | | rs1877660 | |
| 5461384 G | A | G | G | | rs2733360 | |
| 5461402 A | C | A | A | | rs2716917 | |
| 5461446 G | G and A | G | G | | rs35284216 | |
| 5461471 T | C | T | T | | rs2733359 | |
| 5461719-22 | delATGA | ATGA | ATGA | | rs35658367 | |
| ATGA | | | | | | |
| 5461751 A | G | A | A | | rs2716916 | |
| 5461837 A | A and T | A | A | | novel | q = 1/30 |
| 5461916 G | A | G | G | | rs2716915 | |
| 5461934 A | G | A | A | | rs2733358 | |
| 5462473 G | G | G | A | | rs4790785 | |
| 5462497 T | T | T | T | C (2?) | novel | |
| 5462709 G | T | G | G | | rs2733357 | |
| 5462773 A | G | A | A | | rs2733356 | |
| 5462809 A | G | A | A | | rs2733355 | |
| 5463008 T | G | T | T | | rs2716914 | |
| 5463039 G | G and A | G | G | | novel | q = 2/30 |
| 5463125 C | T | C | C | | rs2716913 | |
| 5463606 T | C | T | C | | rs2670662 | |
| 5463878 G | G | G | A | | novel | q = 2/26 |
| 5464579 C | C | G | C | | rs1877661 | |
| 5464977 A | A | A | C | | rs34293306 | |
| 5465892 G | G and A | G | G | | rs34398315 | |
| 5466127 T | G | T | T | | rs2733349 | |
| 5466270 C | A | C | A | | rs4790787 | |
| 5466297 T | T | T | T | C (4?) | novel | q = 1/30 |
| 5466433 C | T | C | C | | rs2716912 | |
| 5466528 A | T | A | T | | rs2670663 | |
| 5466714 G | A | G | A | | novel | q = 1/26 |
| 5466866.IN | G | A | A | | novel | q = 4/30 |
| S99 | | | | | | |
| 5466866.IN | T | T | C | | novel | q = 2/30 |
| S473 | | | | | | |
| 5467571 C | C | C | T | | rs4145810 | |
| 5467792 A | A and C | A | A | | novel | q = 1/30 |
| 5467814 C | T | C | C | | rs12953185 | |
| 5468308 G | G | G | A | | rs35052285 | |
| 5468744 T | insT | T | insT | | novel | q = 2/30 |
| 5468821 G | A and G | G | G | | rs4790791 | |
| 5468865 C | G | C | C | | rs4790792 | |
| 5469201 T | T | T | T | A (4?) | novel | q = 1/30 |
| 5469488 G | C | G | C | | rs2245090 | |
| 5470249 G | G | G | A | | rs11652873 | |
| 5471824 A | C | A | C | | rs2716941 | |
| 5471857 G | G | A | G | | novel | q = 1/26 |
| 5471928 T | T | C | T | | novel | q = 1/26 |
| 5472028 T | C | T | C | | rs7209193 | |
| 5472105 C | C and T | C | C | | novel | q = 1/28 |
| 5472123 T | T | T | C | | rs7209367 | |
| 5472664 C | C and T | C | C | | novel | q = 17/28 |
| 5472849 T | T and A | T | A | | rs907874 | |
| 5473096 G | delG | G | delG | | rs5819034 | |
| 5473479 G | A | G | G | | rs2733380 | |
| 5474169 C | C and T | C | C | | novel | q = 1/30 |
| 5474234 C | A | C | C | | rs2733379 | |
| 5474527 T | T | T | G | | rs907876 | |
| 5475073 T | C | T | T | | rs12185249 | |
| 5475136 G | G | G | A | | rs4790793 | |
| 5475361 A | G | A | A | | rs2670641 | |
| 5475769 C | T | C | T | | rs2255534 | |
| 5475789 T | C | T | C | | rs2175826 | |
| 5475933 A | G | A | G | | rs2137721 | |
| 5476212 C | C | C | G | | rs12946126 | |
| 5476268 A | G | A | A | | rs2670645 | |
| 5476652 A | G | A | A | | rs2670646 | |
| 5477022 A | G | A | A | | rs12453661 | |
| 5477057 G | A | G | A | | rs12450724 | |
| 5477229 A | A and T | A | A | | rs28560347 | |
| 5477258-5477241 | ? | ? | ? | micro- | rs34574375 | |
| (CT)n | | | | satellite | | |
| 5477280 G | A | G | A | | rs2670647 | |
| 5477390 A | G | A | G | | rs2733378 | |
| 5477439 T | A | T | A | | rs2670648 | |
| 5477558 A | T | A | T | | rs2733377 | |
| 5478473-4 | TT | TT | delTT | | rs34444169 | |
| TT | | | | | | |
| 5478658 A | G | A | G | | rs2733346 | |
| 5478791 C | T | C | T | | rs2716924 | |
| 5479111 T | C | T | T | | rs9895574 | |
| 5479553 T | C | T | C | | rs2716923 | |
| 5480430 G | A | G | G | | novel | q = 4/30 |
| 5480758 A | T | A | A | | rs2953436 | |
| 5481027 C | T | C | T | | rs2716942 | |
| 5482009 T | C | T | C | | rs2670650 | |
| 5482301 T | insT | T | insT | | rs36103450 | |
| 5482535 C | C and T | C | C | | novel | q = 1/28 |
| 5482848 C | T | C | T | | rs2733361 | |
| 5483296 A | C | A | C | | rs2716943 | |
| 5483388 G | C | G | C | | rs2670652 | |
| 5483833 A | A and G | A | A | | novel | q = 1/28 |
| 5485397 G | G and A | G | G | | novel | q = 2/28 |
| 5487349 T | C | T | C | | rs8080521 | |
| 5487689 G | A | G | A | | rs7224241 | |
| 5487877 T | G | T | G | | rs7209554 | |
| 5487912 G | G and A | G | G | | novel | q = 1/30 |
| 5488104 C | C and A | C | C | | rs925599 | |
| 5488188 T | C | C | C | | rs4790795 | |
| 5488398 G | G | G | A | | rs4790271 | |
| 5488716 T | G and T | T | T | | rs11656006 | |
| 5488844 T | T | T | insAAAT | | rs34463923 | |
| 5488923 C | C and T | C | C | | rs12450313 | |
| 5488986 A | G | A | A | | rs2716883 | |
| 5489654 A | G | A | A | | rs11078577 | |
| 5489972 A | A | A | G | | rs7215168 | |
| 5490324 G | A | G | G | | rs2203296 | |
| 5490349 A | A and G | A | A | | rs10459902 | |
| 5490607 T | delT | T | T | | rs34343526 | |
| 5490898 G | A | G | G | | rs1513837 | |
| 5491016 T | T and A | T | T | | rs11078578 | |
| 5491023 G | G and A | G | G | | rs1513838 | |
| 5491354 G | G and A | G | G | | novel | q = 18/30 |
| 5491366 T | T and C | T | T | | novel | q = 18/30 |
| 5491405 T | T and C | T | T | | novel | q = 18/30 |
| 5492947 A | G | A | G | | rs2716872 | |
| 5493078 A | G | A | G | | rs2716871 | |
| 5493361 C | C | C | T | | rs4528626 | |
| 5493457 A | A | A | G | | rs4997550 | |
| 5493628 A | A and G | A | A | | rs2670667 | |
| 5493634 C | C and T | C | C | | rs2670666 | |
| 5493686 G | G and C | G | G | | rs2309400 | |
| 5493974 C | G | C | C | | rs878329 | |
| 5494565 T | C | T | T | | rs12452793 | |
| 5494572 C | A | C | C | | rs12450258 | |
| 5494612 A | G | A | A | | rs2871222 | |
| 5494689 A | G | A | A | | rs12453329 | |
| 5494863 G | A | G | G | | rs2018735 | |
| 5495192 A | G | A | A | | rs7223628 | |
| 5495420 T | C | T | T | | rs12945848 | |
| 5495428 C | T | C | C | | rs7217978 | |
| 5495461 G | A | G | G | | rs12451041 | |
| 5495496 G | G | G | A | | rs2037041 | |
| 5495711A | G | A | A | | rs8182352 | |
| 5495986 C | C and T | C | C | | novel | q = 4/30 |
| 5496043 G | A | G | G | | rs4790796 | |
| 5496081 C | T | C | C | | rs4790797 | |
| 5496099 A | G | A | A | | rs4790272 | |
| 5496106 A | G | A | A | | rs4790273 | |
| 5496108 T | C | T | T | | rs4790274 | |
| 5496304 G | A | G | G | | rs8182354 | |

Haplotypes 1, 2, and 3 were defined by the three initially associated Illumina SNPs, rs3926687-rs2733359-rs878329
*For each variant the ENSEMBL nucleotide is considered wild-type, and the observed frequency of the variant given. 12 bp del = nt 5457169-5457180; delTATGACTATGTG

TABLE 9
⁴P Values for Allelic Association of NALP1 Region Variants with Vitiligo and
Extended Autoimmune/Autoinflammatory Disease Phenotype

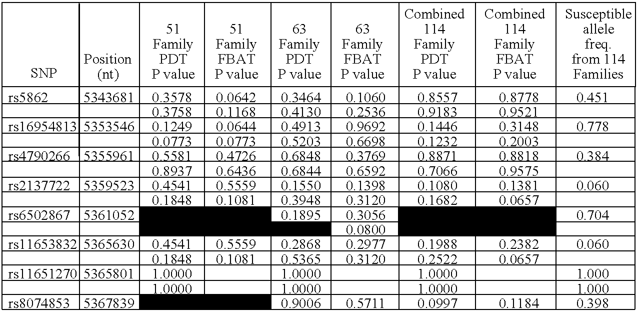

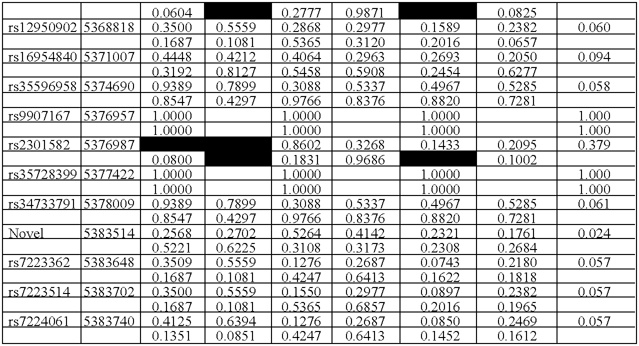

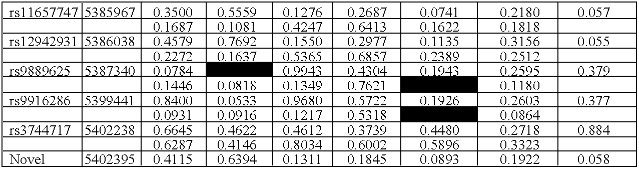

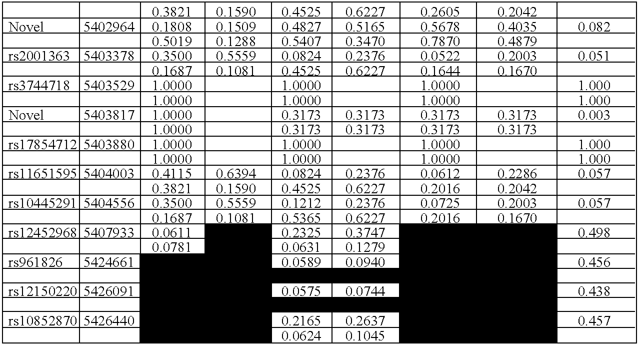

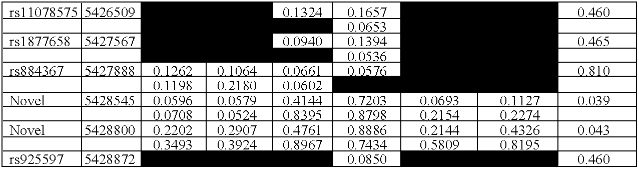

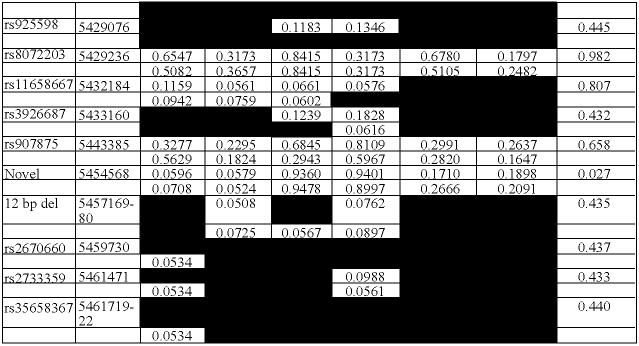

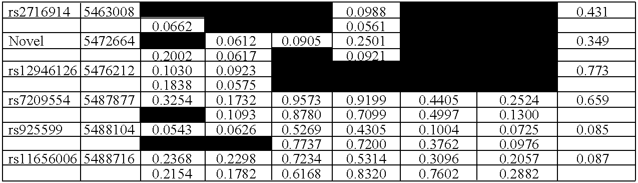

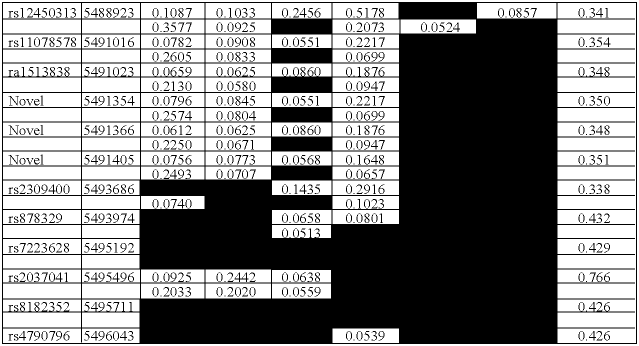

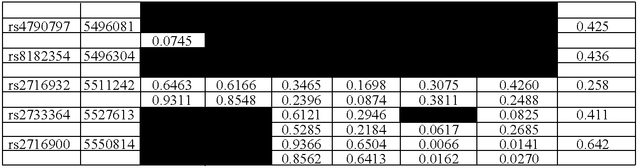

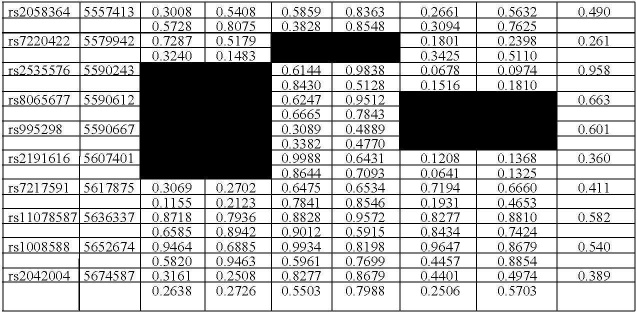

Regular type: vitiligo phenotype;
Italicized type: extended autoimmune/autoinflammatory disease phenotype.
Shaded cells: nominal P value <0.05.
12 bp del = nt 5457169-5457180; delTATGACTATGTG.

TABLE 10
Second locus effect tests in a forward stepwise-regression procedure for the
autoimmune/autoinflammatory disease phenotype

| Null | Alternative | | Null | Alternative | | Null | Alternative | | Null | Alternative | |
|---|---|---|---|---|---|---|---|---|---|---|---|
| Model | Model | P_{clogit} | Model | Model | P_{clogit} | Model | Model | P_{clogit} | Model | Model | P_{clogit} |
| . . . | A | 0.0006 | R | R + A | 0.0068 | . . . | . . . | . . . | A | A + R | 0.0027 |
| A | A + B | 0.0345 | R | R + B | NS | B | B + A | 0.0036 | B | B + R | 0.0247 |
| A | A + C | 0.0311 | R | R + C | NS | C | C + A | 0.0097 | C | C + R | NS |
| A | A + D | NS | R | R + D | NS | D | D + A | 0.0033 | D | D + R | 0.0119 |
| A | A + E | NS | R | R + E | NS | E | E + A | 0.0023 | E | E + R | 0.0027 |
| A | A + F | 0.0310 | R | R + F | NS | F | F + A | 0.0041 | F | F + R | 0.0310 |
| A | A + G | 0.0462 | R | R + G | NS | G | G + A | 0.0099 | G | G + R | 0.0416 |
| A | A + H | 0.0439 | R | R + H | NS | H | H + A | 0.0065 | H | H + R | 0.0328 |
| A | A + I | NS | R | R + I | NS | I | I + A | 0.0034 | I | I + R | 0.0165 |
| A | A + J | 0.0175 | R | R + J | NS | J | J + A | 0.0047 | J | J + R | NS |
| A | A + K | 0.0216 | R | R + K | NS | K | K + A | 0.0030 | K | K + R | 0.0444 |
| A | A + L | 0.0262 | R | R + L | NS | L | L + A | 0.0044 | L | L + R | 0.0414 |
| A | A + M | 0.0490 | R | R + M | NS | M | M + A | 0.0021 | M | M + R | 0.0076 |
| A | A + N | 0.0166 | R | R + N | not | N | N + A | 0.0038 | N | N + R | not |
| | | | | | concave | | | | | | concave |
| A | A + O | 0.0091 | R | R + O | not | O | O + A | 0.0043 | O | O + R | not |
| | | | | | concave | | | | | | concave |
| A | A + P | 0.0034 | R | R + P | not | P | P + A | 0.0067 | P | P + R | not |
| | | | | | concave | | | | | | concave |
| A | A + Q | 0.0066 | R | R + Q | not | Q | Q + A | 0.0053 | Q | Q + R | not |
| | | | | | concave | | | | | | concave |
| A | A + R | 0.0027 | . . . | R | 0.0003 | R | R + A | 0.0068 | . . . | . . . | . . . |
| A | A + S | 0.0104 | R | R + S | NS | S | S + A | 0.0060 | S | S + R | 0.0366 |

SUPPLEMENTARY TABLE 10B
Second locus effect tests in a forward stepwise-regression
procedure for the vitiligo phenotype

| Null | Alternative | | Null | Alternative | | Null | Alternative | | Null | Alternative | |
|---|---|---|---|---|---|---|---|---|---|---|---|
| Model | Model | P_{clogit} | Model | Model | P_{clogit} | Model | Model | P_{clogit} | Model | Model | P_{clogit} |
| . . . | A | 0.0006 | R | R + A | 0.0116 | . . . | . . . | . . . | A | A + R | 0.0028 |
| A | A + B | 0.0471 | R | R + B | NS | B | B + A | 0.0051 | B | B + R | 0.0135 |
| A | A + C | NS | R | R + C | NS | C | C + A | 0.0105 | C | C + R | 0.0217 |
| A | A + D | NS | R | R + D | NS | D | D + A | 0.0043 | D | D + R | 0.0067 |
| A | A + E | NS | R | R + E | NS | E | E + A | 0.0034 | E | E + R | 0.0027 |
| A | A + F | 0.0428 | R | R + F | NS | F | F + A | 0.0059 | F | F + R | 0.0162 |
| A | A + G | NS | R | R + G | NS | G | G + A | 0.0104 | G | G + R | 0.0170 |
| A | A + H | NS | R | R + H | NS | H | H + A | 0.0065 | H | H + R | 0.0112 |
| A | A + I | 0.0496 | R | R + I | NS | I | I + A | 0.0054 | I | I + R | 0.0170 |
| A | A + J | 0.0177 | R | R + J | NS | J | J + A | 0.0069 | J | J + R | NS |
| A | A + K | 0.0213 | R | R + K | NS | K | K + A | 0.0041 | K | K + R | 0.0336 |
| A | A + L | 0.0186 | R | R + L | NS | L | L + A | 0.0075 | L | L + R | 0.0475 |
| A | A + M | 0.0329 | R | R + M | NS | M | M + A | 0.0030 | M | M + R | 0.0115 |
| A | A + N | 0.0198 | R | R + N | not | N | N + A | 0.0050 | N | N + R | not concave |
| | | | | | concave | | | | | | |
| A | A + O | 0.0104 | R | R + O | not | O | O + A | 0.0066 | O | O + R | not concave |
| | | | | | concave | | | | | | |
| A | A + P | 0.0036 | R | R + P | not | P | P + A | 0.0115 | P | P + R | not concave |
| | | | | | concave | | | | | | |
| A | A + Q | 0.0104 | R | R + Q | not | Q | Q + A | 0.0066 | Q | Q + R | not concave |
| | | | | | concave | | | | | | |
| A | A + R | 0.0028 | . . . | R | 0.0002 | R | R + A | 0.0116 | . . . | . . . | . . . |
| A | A + S | 0.0124 | R | R + S | NS | S | S + A | 0.0097 | S | S + R | 0.0259 |

A, rs6502867;
B, rs961826;
C, rs12150220;
D, rs11078575;
E, rs1877658;
F, rs925597;
G, rs925598;
H, rs3926687;
I, 12 bp del (nt 5457169-5457180 delTATGACTATGTG (SEQ ID NO: 1));
J, rs2670660;
K, rs2733359;
L, rs35658369;
M, rs2716914;
N, rs878329;
O, rs7223628;
P, rs8182352;
Q, rs4790796;
R, rs4790797;
S, rs8182354.
P_{clogit}, P values from conditional logistic regression analyses.
NS, not significant (P > 0.05).
“Not concave” means it is impossible to obtain a unique estimate of regression coefficients with both variables in the model, which happens when one independent variable is a perfect linear combination of the other, as in the case of perfect LD.

TABLE 11
Two-SNP haplotype ORs and frequencies in case-pseudocontrol
samples derived from 114 families

| | Total | | | | |
|---|---|---|---|---|---|
| Allele | number | Frequency | P_{cLogit} | OR | 95% C.I |
| hap_1: A.T | 274 | 0.366 | 0.0002 | 2.77 | 1.59-4.81 |
| | 324 | 0.373 | 0.0003 | 2.55 | 1.56-4.17 |
| hap_2: A.C | 294 | 0.393 | 0.1041 | 1.56 | 0.91-2.67 |
| | 329 | 0.379 | 0.0550 | 1.57 | 0.99-2.50 |
| hap_3: G.T | 49 | 0.039 | 0.1553 | 1.76 | 0.81-3.84 |
| | 64 | 0.074 | 0.1080 | 1.64 | 0.90-2.99 |
| hap_4: G.C | 131 | 0.175 | | 1.00 | |
| | 151 | 0.174 | | 1.00 | |

Bold: vitiligo phenotype;
Italicized: extended autoimmune/autoinflammatory disease phenotype.
The two SNPs we used for haplotype construction are rs6502867(A/G SNP, A is the high risk allele) and rs4790797(T/C SNP, T is the high risk allele), each of which represents one of the two independent association signals in the NALP1 region. We used hap_4 as reference haplotype.
P_{cLogit}: P values from conditional logistic regression analyses;
Odds ratios (OR) were calculated from the coefficients of the regression equation.

What is claimed is:

1. An oligonucleotide comprising at least a portion of a nucleic acid sequence of NACHT leucine-rich-repeat protein 1 (NALP1) gene, NALP1 promoter region or combination thereof, the nucleic acid sequence having one or more genetic variations associated with vitiligo or vitiligo-associated autoimmune/autoinflammatory disease (VAAAD) selected from the group consisting of rs6502867, rs961826, rs12150220, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO:1) or their reverse complement), rs2670660, rs2733359, rs35658367, rs2716914, rs878329, rs7223628, rs8182352, rs4790796, rs4790797, rs8182354, SNPs identified by DNA sequence analysis (as listed in Table 8), and a combination thereof.

2. The oligonucleotide of claim 1, wherein the genetic variations comprise one or more of rs3926687, rs2733359 and rs878329.

3. The oligonucleotide of claim 1, wherein the genetic variations comprise Haplotype 1 comprising rs3926687, rs2733359 and rs878329.

4. The oligonucleotide of claim 1, wherein the genetic variations comprise rs470797 and one or more of rs878329, rs7223628, rs8182352, or rs4790796.

5. The oligonucleotide of claim 1, wherein the genetic variations comprise rs2670660/C, rs878329/G, re7223628/G, rs8182352/G, rs4790797/T or combination thereof.

6. The oligonucleotide of claim 1, wherein the nucleic acid sequence contains 2 or more, 3 or more, 4 or more, 5 or more, 6 or more, 7 or more, 8 or more, 9 or more, or 10 or more of the genetic variations.

7. The oligonucleotide of claim 1, wherein the genetic variations comprise one or more of rs961826, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2733359, rs35658367, rs2716914, and rs8182352; and one or more of rs2670660, rs878329, rs7223628, rs8182352, rs4790796, and rs4790797.

8. The oligonucleotide of claim 1, wherein the genetic variations comprise rs6502867 and at least one of rs961826, rs12150220, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2670660, rs2733359, rs35658367, rs2716914, rs878329, rs7223628, rs8182352, rs4790796, rs4790797, or rs8182354.

9. The oligonucleotide of claim 1, wherein the genetic variations comprise rs6502867; and at least one of rs12150220, rs2670660, rs878329, rs7223628, rs8182352, rs4790796, or rs4790797.

10. The oligonucleotide of claim 1, wherein the genetic variations comprise rs6502867, rs12150220 or combination thereof.

11. The oligonucleotide of claim 1, wherein the genetic variation comprises rs12150220 with a substitution of histidine for leucine at position 155 (Leu155→His).

12. A probe comprising an oligonucleotide capable of binding at least a portion of a nucleic acid sequence of the NALP1 gene, NALP1 promoter region or combination thereof comprising, one or more genetic variations associated with vitiligo or vitiligo-associated autoimmune/autoinflammatory disease (VAAAD) selected from the group consisting of rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO:1)), rs2670660/C, rs2733359/G, rs35658367/ATGA, rs2716914/C, rs878329/G, rs7223628/G, rs8182352/G, rs4790796/A, rs4790797/T, rs8182354/A, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’; any SNP listed in Table 9; any two-SNP haplotype as listed in Table 11 or the reverse complement of any of these variations, and a combination thereof.

13. The probe of claim 12, wherein the genetic variations comprise one or more of rs3926687, rs2733359, rs878329, rs12150220 with a substitution of histidine for leucine at position 155 (Leu155→His) or a complementary sequence thereof.

14. The probe of claim 12, wherein the genetic variations comprise rs6502867; and at least one of rs12150220, rs2670660, rs878329, rs7223628, rs8182352, rs4790796, or rs4790797.

15. A method for assessing the risk of at least one of vitiligo or vitiligo-associated autoimmune/autoinflammatory disease (VAAAD) comprising detecting the presence of one or more genetic variations in the NALP1 gene, NALP1 promoter region or combination thereof.

16. The method of claim 15, wherein the one or more genetic variations are selected from the group consisting of rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1)), rs2670660/C, rs2733359/G, rs35658367/ATGA, rs2716914/C, rs878329/G, rs7223628/G, rs8182352/G, rs4790796/A, rs4790797/T, rs8182354/A, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’; any SNP listed in Table 9; any two-SNP haplotype as listed in Table 11 or the reverse complement of any of these variations, the complement thereof and a combination thereof.

17. The method of claim 15, wherein the genetic variations comprise rs6502867; and at least one of rs12150220, rs2670660, rs878329, rs7223628, rs8182352, rs4790796, or rs4790797.

18. The method of claim 15, wherein the risk of vitiligo or VAAAD increases with a combination of genetic variations selected from the group consisting of one or more of rs961826, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2733359, rs35658367, rs2716914, and rs8182352; and one or more of rs2670660, rs878329, rs7223628, rs8182352, rs4790796, and rs4790797.

19. The method of claim 15, wherein the VAAAD is selected from the group consisting of generalized vitiligo, autoimmune thyroid disease, adult-onset autoimmune diabetes mellitus (also known as latent autoimmune diabetes in adults, LADA, and type 1.5 diabetes mellitus), rheumatoid arthritis, psoriasis, pernicious anemia, systemic lupus erythematosus, Addison's disease and a combination thereof.

20. A method for diagnosing at least one of vitiligo or any of the component disorders of VAAAD comprising detecting the presence of one or more genetic variations in the NALP1 gene, NALP1 promoter region or combination thereof, the variations associated with risk of VAAAD.

21. The method of claim 20, wherein the one or more genetic variations are selected from the group consisting of rs6502867/A, rs961826/A, rs12150220/A, rs11078575/C, rs1877658/T, rs925597/A, rs925598/A, rs3926687/T, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2670660/C, rs2733359/G, rs35658367/ATGA, rs2716914/C, rs878329/G, rs7223628/G, rs8182352/G, rs4790796/A, rs4790797/T, rs8182354/A, or the reverse complement of any of these variations, and a combination thereof.

22. The method of claim 20, wherein the genetic variations comprise rs6502867; and at least one of rs12150220, rs2670660, rs878329, rs7223628, rs8182352, rs4790796, or rs4790797.

23. A method for assessing the risk of adult-onset autoimmune diabetes mellitus (also known as latent autoimmune diabetes in adults, LADA, and type 1.5 diabetes mellitus) in a subject comprising detecting the presence of one or more genetic variations in the NALP1 gene, NALP1 promoter region or combination thereof, the variations associated with risk of at least one of adult-onset autoimmune diabetes mellitus or any of the component disorders of VAAAD.

24. The method of claim 23, wherein the one or more genetic variations are selected from the group consisting of rs6502867, rs8074853, rs16954840, rs12150220, rs2670660, rs8182352, rs11078587, rs1008588 and a combination thereof.

25. The method of claim 23, wherein the genetic variations comprise rs6502867; and at least one of rs12150220, rs2670660, rs878329, rs7223628, rs8182352, rs4790796, or rs4790797.

26. A method of treating a subject for at least one of vitiligo or any of the component disorders of VAAAD comprising administering to the subject in need of such a treatment, a therapeutically effective amount of a composition targeting one or more genetic variations in NALP1 gene, NALP1 promoter region or combination thereof.

27. The method of claim 26, wherein the genetic variations are selected from the group consisting of rs6502867, rs961826, rs12150220, rs11078575, rs1877658, rs925597, rs925598, rs3926687, a deletion at nucleotide (nt) 5,457,169-5,457,180 of the National Center for Biotechnology Information Build 36 human chromosome 17 DNA sequence (nucleotides 5′-TATGACTATGTG-3′ (SEQ ID NO: 1) or their reverse complement), rs2670660, rs2733359, rs35658367, rs2716914, rs878329, rs7223628, rs8182352, rs4790796, rs4790797, rs8182354, haplotype 1, haplotype 2, haplotype 3, any SNP listed in Table 2; any SNP as listed in Table 3; any SNP listed in Table 4; any SNP listed in Table 6; any SNP listed in Table 7; any SNPs carried on a haplotype as listed in Table 8 columns Haplotype 1, Haplotype 2, Haplotype 3 and ‘unknown’; any SNP listed in Table 9; any two-SNP haplotype as listed in Table 11 or the reverse complement of any of these variations, and a combination thereof.

28. The method of claim 26, wherein the composition ameliorates a symptom of at least one of vitiligo or any of the component disorders of VAAAD.

29. The method of claim 26, wherein the composition reduces the risk of developing at least one of vitiligo or any of the component disorders VAAAD.

30. The method of claim 26, wherein the composition reduces progression of at least one of vitiligo or any of the component disorders VAAAD.

31. The method of claim 26, wherein the composition further comprises an interleukin-1β inhibitor, an interleukin-1β receptor antagonist, an interleukin-1β receptor inhibitor, a caspase inhibitor, an interleukin-18 antagonist, an interleukin-18 inhibitor, an ASC antagonist, an ASC inhibitor, or combination thereof.

32. The method of claim 26, wherein the composition further comprises IL1-RA, Kinaret, Anikinra, ASC1, ASC-1, PYD and CARD domain containing protein (PYCARD) or a combination thereof.

33. A method of inhibiting apoptosis comprising administering a therapeutic agent targeted to NALP1 or a caspase, wherein said administering inhibits apoptosis.

34. The method of claim 33, wherein the agent is administered to one or more cells.

35. The method of claim 33, wherein the agent is administered to a subject.

36. The method of claims 33, wherein the subject is a human subject.

37. The method of claims 33, further comprising testing for the presence of one or more genetic variations in NALP1 before administering the agent.

38. A kit comprising:
a container means; and
at least one probe capable of binding rs6502867 of the NALP1 gene.

39. The kit of claim 38, further comprising one or more additional probes capable of binding to rs12150220, rs2670660, rs878329, rs7223628, rs8182352, rs4790796, or rs47907970.
